SEQUENCE LISTING (32 sequences)

SEQ ID NO: 1 - protein, 31 aa, Artificial Sequence
  TIP 9-39
AFRERARLLAALERRHWLNSYMHKLLVLDAP

SEQ ID NO: 2 - protein, 37 aa, Artificial Sequence
  TIP 3-39
ALADDAAFRERARLLAALERRHWLNSYMHKLLVLDAP

SEQ ID NO: 3 - protein, 32 aa, Artificial Sequence
  TIP 8-39
AAFRERARLLAALERRHWLNSYMHKLLVLDAP

SEQ ID NO: 4 - protein, 30 aa, Artificial Sequence
  TIP 10-39
FRERARLLAALERRHWLNSYMHKLLVLDAP

SEQ ID NO: 5 - protein, 29 aa, Artificial Sequence
  TIP 11-39
RERARLLAALERRHWLNSYMHKLLVLDAP

SEQ ID NO: 6 - protein, 28 aa, Artificial Sequence
  TIP 12-39
ERARLLAALERRHWLNSYMHKLLVLDAP

SEQ ID NO: 7 - protein, 39 aa, Artificial Sequence
  bTIP39
SLALADDAAFRERARLLAALERRHWLNSYMHKLLVLDAP

SEQ ID NO: 8 - protein, 37 aa, Artificial Sequence
  hPTH
SVSEIQLMHNLGKHLNSMERVEWLRKKLQDVHNFVAL

SEQ ID NO: 9 - protein, 37 aa, Artificial Sequence
  bPTH
AVSEIQFMHNLGKHLSSMERVEWLRKKLQDVHNFVAL

SEQ ID NO: 10 - protein, 37 aa, Artificial Sequence
  bPTP
AVSEHQLLHDKGKSIQDLRRRFFLHHLIAEIHTAEIR

SEQ ID NO: 11 - protein, 37 aa, Artificial Sequence; identical to SEQ ID NO: 10
  hPTP

SEQ ID NO: 12 - protein, 37 aa, Artificial Sequence
  PTP(1-20)/TIP(23-39)
AVSEHQLLHDKGKSIQDLRRRHWLNSYMHKLLVLDAP

SEQ ID NO: 13 - protein, 37 aa, Artificial Sequence
  PTP(1-9)/TIP(12-39)
AVSEHQLLHERARLLAALERRHWLNSYMHKLLVLDAP

SEQ ID NO: 14 - protein, 37 aa, Artificial Sequence
  PTP(1-13)/TIP(16-39)
AVSEHQLLHDKGKLLAALERRHWLNSYMHKLLVLDAP

SEQ ID NO: 15 - DNA, 21 bp, Artificial Sequence
  hTIPr5 Primer
agcagcttgtgcatgtacgag

SEQ ID NO: 16 - DNA, 21 bp, Artificial Sequence
  hTIPr4 Primer
ttgtgcatgtacgagttcagc

SEQ ID NO: 17 - DNA, 22 bp, Artificial Sequence
  mTIP2rev Primer
gtccagtagcaacagcttctgc

SEQ ID NO: 18 - DNA, 22 bp, Artificial Sequence
  mTIPCR2-f6 Primer
tctctatttttatccctctgac

SEQ ID NO: 19 - DNA, 21 bp, Artificial Sequence
  mTIPCR2-f5 primer
ctctgacacaccccttgtgtc

SEQ ID NO: 20 - DNA, 23 bp, Artificial Sequence
  mTIP5for Primer
ctagctgacgacgcggcctttcg

SEQ ID NO: 21 - DNA, 9 bp, Mus sp. and Homo sapiens
  Combined DNA/RNA Sequence
cggtgaugg

SEQ ID NO: 22 - DNA, 14 bp, Homo sapiens
actcacggtagggg

SEQ ID NO: 23 - DNA, 16 bp, Homo sapiens
tctccacaggtgatgg

SEQ ID NO: 24 - DNA, 14 bp, Homo sapiens
tcctcaggtaggtg

SEQ ID NO: 25 - DNA, 16 bp, Homo sapiens
tctccacagcctccgc

SEQ ID NO: 26 - DNA, 14 bp, Mus sp.
ctgcacggtagggg

SEQ ID NO: 27 - DNA, 14 bp, Mus sp.
tccacaggtgatgg

SEQ ID NO: 28 - DNA, 14 bp, Mus sp.
tgttcaggtaggtg

SEQ ID NO: 29 - DNA, 14 bp, Mus sp.
cccccagcctccgc

SEQ ID NO: 30 - DNA, 1440 bp, Homo sapiens
  5′UTR (73)..(324): Exon U1
tttgtgtctctgttcctctgacggtccttccgccagtctctatttttagccctctgacacaccccctgtgtccacctctctgtctgtctgtctctccccctccctcgtctcaggtccagcttctggtcccaattagttggtggcggccaaggcagcggcaggtcccccacccccggctcctcattaccgctggcggctcctaatgagcctggggagggggtgaccccgcgtccccggccccccggcctgcgtcactgcccggtgcgggggctgcggaggcgatataagggggctgccaccatcgctgccccagcccactgcacggtaggggactgtgcgggaagctgggggtggatgcatggtggggcccggggttctgggccgggatgcagccctactgagcccctttctggttctccacaggtgatggagacccgccaggtgtccaggagccctcgggttcggctgctgctgctgctgctgctgctgctggtggtgccctggggcgtccgcactgcctcgggagtcgccctgcccccggtcggggtcctcaggtaggtgccagtcccagaactcccagggaggggtgggaacttggagaagtgggaagagaaccaaagagaaaggggacggaagatccagaaagcggaacagaagcccaaagagagggcgaccgagacccagcgagaagacagagactaggggtgggggaaggcgggaggagggtttggtggtagtggtggtgcgggaaatggacagaaaatcagcaagggaaagagacacagaaagatagatcccgagaaaggcgtggagaggctcgaaaggagtcgttgcagaaacggattcattctttgagcaacgtttattgggttcttggtgtgcgcctggccatgcgctgagcgctgtgggcggacaagtgcatcagactcggtctcagtacctggggagtttcgaacctggtgagcagagagacagagacccacacagagaacgagagagacagggggagccggaccgagccactccacggactgggcagtaacggggcttcgagttagaccgcggggaggacaggcggcggcaagagcgagtctggacgcgcggtcaccgcgtctctccacagcctccgccccccaggacgggcctgggcggatcccgccacccccaggccgcggaggagcctggcgctggcggacgacgcggccttccgggagcgcgcgcggttgctggccgccctcgagcgccgccactggctgaactcgtacatgcacaagctgctggtgttggatgcgccctgagcgcgctgcccgtccccatcttaataaagaccatgccctgcgctccggactgcgcctcgttcctgcgcgacctgcgtgtgcgttgggttgggggcgcggggcttgaaatggggggtacaaaagagacacgactctgtgtcgg

SEQ ID NO: 31 - protein, 100 aa, Homo sapiens
METRQVSRSPRVRLLLLLLLLLVVPWGVRTASGVALPPVGVLRLRPPGRAWADPATPRPRRSLALADDAAFRERARLLAALERRHWLNSYMHKLLVLDAP

SEQ ID NO: 32 - protein, 100 aa, Mus sp.
METCQMSRSPRERLLLLLLLLLLVPWGTGPASGVALPLAGVFSLRAPGRAWAGLGSPLSRRSLALADDAAFRERARLLAALERRRWLDSYMQKLLLLDAP

(19) United States
(12) Patent Application Publication
(10) Pub. No.: US 2004/0176285 A1
(43) Pub. Date: Sep. 9, 2004

(54) Tip39 polypeptides

(76) Inventors: Harald Juppner, Cambridge, MA (US); Thomas J Gardella, Needham, MA (US); Kenneth P Jonsson, Uppsala (SE); Markus R John, Brookline, MA (US); Robert C Gensure, Luling, LA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/466,483

(22) PCT Filed: Jan. 17, 2002
(86) PCT No.: PCT/US02/01183

Related U.S. Application Data
(60) Provisional application No. 60/261,804, filed on Jan. 17, 2001.

(51) Int. Cl. A61K 38/17; C07K 7/08

(52) U.S. Cl. 514/12; 530/324; 536/23.5

(57) ABSTRACT

The invention relates to truncated TIP39 polypeptides and chimeric PTHrP/TIP polypeptides. The polypeptides are used as agonists or antagonists of PTH receptors in various medical conditions.

### BACKGROUND OF THE INVENTION

1. Field of the Invention

The invention is related to the fields of molecular biology, endocrinology and medicine. In particular, it relates to PTH receptor agonists and antagonists.

2. Background Art

Parathyroid Hormone

Parathyroid hormone (PTH) is a major regulator of calcium homeostasis whose principal target cells occur in bone and kidney. Regulation of calcium concentration is necessary for the normal function of the gastrointestinal, skeletal, neurologic, neuromuscular, and cardiovascular systems. PTH synthesis and release are controlled principally by the serum calcium level; a low level stimulates and a high level suppresses both hormone synthesis and release. PTH, in turn, maintains the serum calcium level by directly or indirectly promoting calcium entry into the blood at three sites of calcium exchange: gut, bone, and kidney. PTH contributes to net gastrointestinal absorption of calcium by favoring the renal synthesis of the active form of vitamin D. PTH promotes calcium resorption from bone indirectly by stimulating differentiation of the bone-resorbing cells, osteoclasts. It also mediates at least three main effects on the kidney: stimulation of tubular calcium reabsorption, enhancement of phosphate clearance, and promotion of an increase in the enzyme that completes synthesis of the active form of vitamin D. PTH exerts these effects primarily through receptor-mediated activation of adenylate cyclase and phospholipase C.

Disruption of calcium homeostasis may produce many clinical disorders (e.g., severe bone disease, anemia, renal impairment, ulcers, myopathy, and neuropathy, hypercalcemia) and usually results from conditions that produce an alteration in the level of parathyroid hormone. Hypercalcemia is a condition that is characterized by an elevation in the serum calcium level. It is often associated with primary hyperparathyroidism in which an excess of PTH production occurs as a result of a lesion (e.g., adenoma, hyperplasia, or carcinoma) of the parathyroid glands. Another type of hypercalcemia, humoral hypercalcemia of malignancy (HHM) is the most common paraneoplastic syndrome. It appears to result in most instances from the production by tumors (e.g., squamous, renal, ovarian, or bladder carcinomas) of a class of protein hormone which shares amino acid homology with PTH. These PTH-related proteins (PTHrP) appear to mimic certain of the renal and skeletal actions of PTH and are believed to interact with the PTH receptor in these tissues. PTHrP is normally found at low levels in many tissues, including keratinocytes, brain, pituitary, parathyroid, adrenal cortex, medulla, fetal liver, osteoblast-like cells, and lactating mammary tissues. In many HHM malignancies, PTHrP is found in the circulatory system at high levels, thereby producing the elevated calcium levels associated with HHM.

The pharmacological profiles of PTH and PTHrP are nearly identical in most in vitro assay systems, and elevated blood levels of PTH (i.e., primary hyperparathyroidism) or PTHrP (i.e., HHM) have comparable effects on mineral ion homeostasis (Broadus, A. E. & Stewart, A. F., “Parathyroid hormone-related protein: Structure, processing and physiological actions,” in Basic and Clinical Concepts, Bilzikian, J. P. et al., eds., Raven Press, New York (1994), pp. 259-294; Kronenberg, H. M. et al., “Parathyroid hormone: Biosynthesis, secretion, chemistry and action,” in Handbook of Experimental Pharmacology, Mundy, G. R. & Martin, T. J., eds., Springer-Verlag, Heidelberg (1993), pp. 185-201). The similarities in the biological activities of the two ligands can be explained by their interaction with a common receptor, the PTH/PTHrP receptor, which is expressed abundantly in bone and kidney (Urena, P. et al., Endocrinology 134:451-456 (1994)).

PTH/PTHrP Receptor

The PTH/PTHrP receptor (also referred to as PTH1R) is activated with equal potency and efficacy by parathyroid hormone (PTH) and PTH-related peptide (PTHrP), two peptides which share only limited amino acid sequence homology (for review see (Gardella, T. J., and Jüppner, H., “Interaction of PTH and PTHrP with their receptors,” in Reviews Endocrine Metabolic Disorders, Kluwer Academic Publisher, The Netherlands (2000), p. 317-329; Jüppner, H., et al., “Parathyroid hormone and parathyroid hormone-related peptide in the regulation of calcium homeostasis and bone development,” in DeGroot, L. J., ed., Endocrinology, W. B. Saunders, Philadelphia, Pa. (969-998 (2000)). The PTH1R is a member of the class B family of G protein-coupled receptors and is expressed in numerous tissues, most abundantly in kidney, bone, and growth plate chondrocytes. By mediating the actions of two distinct peptides, the PTH1R serves multiple biological roles, including the PTH-dependent endocrine regulation of mineral ion homeostasis and bone turnover, and the PTHrP-dependent autocrine/paracrine regulation of endochondral bone formation (for review see (Jüppner, H., et al., “Parathyroid hormone and parathyroid hormone-related peptide in the regulation of calcium homeostasis and bone development,” in DeGroot, L. J., ed., Endocrinology, W. B. Saunders, Philadelphia, Pa. (969-998 (2000)); Lanske, B., and Kronenberg, H., Crit. Rev. Eukaryot. Gene Expr. 8:297-320 (1998)).

In contrast to the firmly established, homeostatic and developmental roles of the PTH IR, the biological role of the PTH2 receptor (also) referred to as PTH2R) remains unknown (Usdin, T. B., et al., Nature Neuroscience 2:941-943 (1999)). Unlike the widely expressed PTH1R, the PTH2R is found only in a few tissues, including the hypothalamus. Although initial functional characterization of the human PTH2R had shown that it is activated by PTH, but not by PTHrP (Usdin, T. B., et al., J. Biol. Chem. 270:15455-15458 (1995); Usdin, T. B., et al., Endocrinology 137:4285-4297 (1996)), subsequent radioreceptor studies revealed that PTHrP binds, although poorly, to the human PTH2R (Gardella, T. J., et al., J. Biol. Chem. 271:19888-19893 (1996); Behar, V., et al., Endocrinology 137:4217-4224 (1996); Clark, J. A., et al., Mol. Endocrinol. 12:193-206 (1998)). The IC₅₀ of PTHrP(1-36) at the PTH2R was increased 7-fold when Phe23 was replaced by Trp, which is found at position 23 in all PTH species. However, despite improved apparent binding affinity, this Trp23-modified analog continued to lack agonist activity at the PTH2R, which implied that the amino-terminus of PTHrP is incompatible with this receptor (Gardella, T. J., et al., J. Biol. Chem. 271:19888-19893 (1996)). When His5 was modified to the PTH-specific residues of isoleucine, the resulting PTHrP(1-36) analog activated the PTH2R with full or nearly full potency (Gardella, T. J., et al., J. Biol. Chem. 271:19888-19893 (1996); Behar, V., et al., Endocrinology 137:4217-4224 (1996)). Conversely, replacement of Ile5 in PTH(1-34) with histidine led to an analog with severely impaired capacity to stimulate cAMP accumulation at the PTH2R, implying that position 5 in either ligand is of critical importance for determining receptor signaling selectivity at this receptor (Gardella, T. J., et al., J. Biol. Chem. 271:19888-19893 (1996)).

Subsequent investigations with [Trp²³]PTHrP(1-36)amide, [Ile⁵, Trp²³]PTHrP(1-36)amide, and reciprocal PTH1R/PTH2R chimeras led to the identification of regions and individual residues in the PTH2R that play an essential role in determining agonist selectivity of this receptor, particularly regarding residue 5 of the ligand (Bergwitz, C., et al., J. Biol. Chem. 272:28861-28868 (1997)). Independently, Turner et al. (Turner, P. R., et al., J. Biol. Chem. 273:3830-3837 (1998)) and Clark et al. (Clark, J. A., et al., Mol. Endocrinol. 12:193-206 (1998)) used receptorchimeras and mutagenesis studies to explore ligand selectivity of the PTH2R. In each of these studies, residues in receptor regions comprising transmembrane helices and extracellular loops were found to be involved in determining agonist selectivity for PTH and PTHrP. The availability of two related but structurally distinct ligands and of two PTH-receptor subtypes that responded differentially to these ligands, thus led to new insights into the molecular determinants of recognition and ligand-dependent activation of the PTH2R.

In contrast to the human PTH2R, which is fully activated by PTH but not by PTHrP, recent data indicated that the rat PTH2R is not responsive to either PTH or PTHrP (Hoare, S. R., et al., Endocrinology 140:44194425 (1999)). These findings suggested that the primary ligand for the PTH2R is not PTH or PTHrP, and indeed partially purified extracts from bovine hypothalamus were shown to contain a peptide that stimulated the human and rat PTH2R, but not the PTH1R (Usdin, T. B., Endocrinology 138:831-834 (1997)). Subsequent studies led to the isolation of TIP39, a 39 amino acid peptide (herein referred to as TIP(1-39)) that efficiently activates the PTH2R homologs from several different species, including zebrafish, but not the PTH1R (Usdin, T. B., et al., Nature Neuroscience 2:941-943 (1999); Hoare, S. R. J., et al., Endocrinology 141:3080-3086 (2000)). The limited amino acid sequence identity shared by TIP(1-39), PTH(1-34), and PTHrP(1-36) is restricted to the carboxyl-terminal region, which contains several conserved residues that have been shown to be functionally important in both latter peptides (FIG. 1). By interacting predominantly with the amino-terminal, extracellular domain of the PTH1R, the carboxyl-terminal region of PTH(1-34) and PTHrP(1-36) plays a principal role in determining high affinity receptor binding, and this interaction is thought to position the amino-terminal domain of either ligand within the region of the receptor that is required for activation (Gardella, T. J., and Jüppner, H., “Interaction of PTH and PTHrP with their receptors,” in Reviews Endocrine Metabolic Disorders, Kluwer Academic Publisher, The Netherlands (2000), p. 317-329; Jüppner, H., et al., Endocrinology 134:879-884 (1994); Adams, A. E., e al., Biochemistry 34:10553-10559(1995); Bergwitz, C., et al., J. Biol. Chem. 271:26469-26472 (1996); Zhou, A. T., et al., Proc. Natl. Acad. Sci. USA 94:3644-3649 (1997); Mannstadt, M., et al., J. Biol. Chem. 273:16890-16896 (1998)).

Accordingly, there is a need in the art for the development of PTH/PTHrP receptor (PTH1R) agonists and antagonists: 1) to assist in further elucidating the role of the PTH/PTHrP receptor; 2) to map specific sites of ligand-receptor interaction; and 3) as potential new therapeutic compositions that can be used in the treatment of disorders having altered action or genetic mutation of the receptor. Furthermore, there is a need in the art for the development of PTH2 receptor (PTH2R) agonists and antagonists: 1) to assist in further elucidating the role of the PTH2 receptor; 2) to map specific sites of ligand-receptor interaction; and 3) as potential new therapeutic compositions that can be used in the treatment of disorders having altered action or genetic mutation of the receptor

### BRIEF SUMMARY OF THE INVENTION

The invention is first directed to an isolated polypeptide consisting of the amino acid sequence AFRERARLLAALERRHWLNSYMHKLLVLDAP. [SEQ ID No.:1] or ALADDAAFRERARLLAALERRHWLNSYMHKLLVLDAP. [SEQ ID No.:2].

The invention is further directed to an isolated polypeptide comprising an amino acid sequence selected from the group consisting of ALADDAAFRERARLLAALERRHWLNSYMHKLLVLDAP. [SEQ ID No.:2), AAFRERARLLAALERRHWLNSYMHKLLVLDAP [SEQ ID No.:3], FRERARLLAALERRHWLNSYMHKLLVLDAP [SEQ ID No.:4], AFRERARLLAALERRHWLNSYMHKLLVLDAP. [SEQ ID No.:1]. RERARLLAALERRHWLNSYMHKLLVLDAP [SEQ ID No.:5] and ERARLLAALERRHW LNSYMHKLLVLDAP [SEQ ID No.:6], wherein said polypeptide sequence is not TIP7-39.

Another aspect of the invention is directed to the isolated polypeptides ALADDAAFRERARLLAALERRHWLNSYMHKLLVLDAP. [SEQ ID No.:2], AAFRERARLLAALERRHWLNSYMHKLLVLDAP [SEQ ID No.:3], FRERARLLAALERRHWLNSYMHKLLVLDAP [SEQ ID No.:4], AFRERARLLAALERRHWLNSYMHKLLVLDAP. [SEQ ID No.:1]. RERARLLAALERRHWLNSYMHKLLVLDAP [SEQ ID No.:5] and ERARLLAALERRHWLNSYMHKLLVLDAP [SEQ ID No.:6] wherein there is a single amino acid substitution. Further embodiments of the invention relate to these isolated polypeptides wherein there is one or more conservative amino acid substitutions. Examples of conservative amino acid substitutions are found in Table 1 of the specification. Additional embodiments of the invention are directed to derivatives of any of the specific sequences of the claimed invention such as for example where there are one or more amino acid substitutions such that the derivative maintains its activity as either an agonist or antagonist of PTH1R or PTH2R.

The invention is further directed to production of antibodies against any of the isolated polypeptides of the invention.

The invention is further directed to an isolated nucleic acid sequence encoding any of the polypeptides of the invention. In this regard, embodiments of the invention are also directed to an isolated nucleic acid sequence, wherein said sequence is at least 95% identical or binds under stringent conditions to the nucleic acid sequences encoding any one of ALADDAAFRERARLL AALERRHWLNSYMHKLLVLDAP. [SEQ ID No.:2], AAFRERARLLAALERRHWLNSYMHKLLVLDAP [SEQ ID No.:3], FRERARLLAALERRHWLNSYMHKLLVLDAP [SEQ ID No.:4], AFRERARLLAALERRHWLNSYMHKLLVLDAP. [SEQ ID No.:]]. RERARLLAALERRHWLNSYMHKLLVLDAP [SEQ ID No.:5] and ERARLLAALERRHW LNSYMHKLLVLDAP [SEQ ID No.:6]

The invention is further directed to recombinant host cells or recombinant vectors comprising DNA encoding any one of ALADDAAFRERARLLA ALERRHWLNSYMHKLLVLDAP. [SEQ ID No.:2], AAFRERARLLAALERR HWLNSYMHKLLVLDAP [SEQ ID No.:3], FRERARLLAALERRHWLNSYMHKLLVLDAP [SEQ ID No.:4], AFRERARLLAALERRHWLNSYMHKLLVLDAP. [SEQ ID No.:1]. RERARLLAALERRHWLNSYMHKLLVLDAP [SEQ ID No.:5] and ERARLLAALERRHWLNSYMHKLLVLDAP [SEQ ID No.:6]

The invention is further directed to an isolated polypeptide, wherein said polypeptide is a truncated polypeptide of TIP39 (SLALADDAAFRERARL LAALERRHWLNSYMHKLLVLDAP) [SEQ ID No.:7] and said truncated polypeptide is not TIP7-39.

J The invention is also directed to a method for treating a mammalian condition in that said condition is characterized by requiring antagonism of PTH1R or PTH2R, said method comprising: a) administering to a patient in need of antagonism of PTHR1R or PTH2R, an effective dose of any of the polypeptides of the invention; and b) antagonizing PTH1R or PTH2R. Preferable embodiments of the invention are directed to treatment of hypercalcemia and hyperparathyroidism. Additional embodiments of the invention are directed to treatment of hyperparathyroidism (PTH-dependent) or humoral hypercalcemia of malignancy (PTHrP-dependent) and to condition mediated by the PTH2R. Preferably the polypeptides are selected from the group ALADDAAFRERARLL AALERRHWLNSYMHKLLVLDAP. [SEQ ID No.:2], AAFRERARLLAALERRHWLNSYMHKLLVLDAP [SEQ ID No.:31, FRERARLLAALERRHWLNSYMHKLLVLDAP [SEQ ID No.:4], AFRERARLLAALERRHWLNSYMHKLLVLDAP. [SEQ ID No.:1]. RERARLLAALERRHWLNSYMHKLLVLDAP [SEQ ID No.:5] and ERARLLAALERRHWLNSYMHKLLVLDAP [SEQ ID No.:6].

Further embodiments of the invention are directed to methods where the effective amount of polypeptide antagonizing PTH1R or PTH2R is administered by providing to the patient DNA encoding said polypeptide and expressing said polypeptide in vivo.

The invention is further directed to an isolated polypeptide comprising the sequence AFRERARLLA, wherein said sequence is not that of the polypeptide TIP39 or TIP7-39 and said isolated polypeptide binds to PTH1R or PTH2R. Such a polypeptide may be used in the methods of treatment of the invention.

The invention is further directed to a PTH1R antagonist comprising a truncated TIP39 polypeptide wherein said antagonist is not TIP7-39. Preferably the PTH1R antagonist is TIP3-39 or TIP9-39. Embodiments of the invention are directed to antagonists with an apparent binding affinity at least 2-fold higher, 3-fold higher and 5 fold higher than TIP1-39.

The invention is further directed to a PTH2R antagonist with an apparent binding affinity that is higher than 1/1000th that of TIP1-39. An additional embodiment is further directed to a PTH2R antagonist with an apparent binding affinity that is higher than 1/100th that of TIP1-39.

The invention is further directed to a PTH1R agonist comprising the sequence of the chimeric polypeptide PTHrP(1-20)/TIP(23-39) (AVSEHQLLHDKGKSIQDLRRRHWLNSYMHKLLVLDAP) [SEQ ID NO:8]. Further aspects of the invention are directed to PTHrP(1-9)/TIP(12-39) (AVSEHQLLHERARLLAALERRHWLNSYMHKLLVLDAP) [SEQ ID NO:13]

and PTHrP(1-13)/TIP(16-39)(AVSEHQLLHDKGKLLAALER RHWLNSYMHKLLVLDAP) [SEQ ID NO:14]. Additional embodiments of the invention are directed to TIP39/PTH or TIP39/PTHrP chimera.

Yet another aspect of the invention is directed to a method for treating mammalian conditions characterized by increases in blood calcium resulting from excess PTH or PTHrP comprising: a) administering to a patient in need thereof an effective dose any one of the polypeptides ALADDAAFRERARLLAALERRHWLNSYMHKLLVLDAP. [SEQ ID No.:2], AAFRERARLLAALERRHWLNSYMHKLLVLDAP [SEQ ID No.:3], FRERARLLAALERRHWLNSYMHKLLVLDAP [SEQ ID No.:4], AFRERARLLAALERRHWLNSYMHKLLVLDAP. [SEQ ID No.:]], RERARLLAALERRHWLNSYMHKLLVLDAP [SEQ ID No.:5] and ERARLLAALERRHWLNSYMHKLLVLDAP [SEQ ID No.:6, and b) antagonizing PTH1R.

The invention is further directed to a method for treating mammalian conditions characterized by decreases in bone mass, wherein said method comprises administering to a subject in need thereof an effective bone mass-increasing amount of the chimeric polypeptide PTHrP(1-20)MP(23-39) (AVSEHQLLHDKGKSIQDLRRRHWLNSYMHKLLVLDAP) [SEQ ID NO: 8] PTHrP(1-9)/TIP(12-39) (AVSEHQLLHERARLLAALER RHWLNSYMHKLLVLDAP) [SEQ ID NO:13] and PTHrP(1-13)/TIP(16-39) (AVSEHQLLHDKGKLLAALERRHWLNSYMHKLLVLDAP) [SEQ ID NO:14]. Another aspect of the invention involves treating the same condition by providing to the patient DNA encoding said peptide and expressing said peptide in vivo. Preferably the condition to be treated may be osteoporosis. Administration of the polypeptide may be by any methods know to those of skill in the art preferably at an effective amount of said polypeptide from about 0.01 μg/kg/day to about 1.0 μg/kg/day.

In accordance with yet a further aspect of the invention, there is provided a method for treating osteoporosis, comprising administering to a patient a therapeutically effective amount of a chimeric polypeptide (PTHrP(1-20)/Tip(23-39), PTHrP(1-9)/TIP(12-39) or PTHrP(1-13)/TIP(16-39) of the invention or a derivative thereof, sufficient to activate the PTHR1 or PTH2R receptor of said patient. Similar dosages and administration as described above for the PTHR1 or PTH2R antagonist, may be used for administration of a PTHR1 agonist, e.g., for treatment of conditions such as osteoporosis, other metabolic bone disorders, and hypoparathyroidism and related disorders. Preferably, the dosage will be 1/10 to 1/100 that of the dosage for the antagonist.

The invention is further directed to a method for healing conditions characterized by an abnormality related to the activated PTH2R.

### BRIEF DESCRIPTION OF THE DRAWINGS/FIGURES

FIG. 1. Amino-terminal amino acid sequences of human and bovine PTH, bovine TIP(1-39), and human and bovine PTHrP. Residues that are identical in PTH and PTHrP are indicated by the shaded area; residues that are conserved between TIP(1-39) and PTH or PTHrP are boxed; numbers indicate the position of the residues in the PTH and PTHrP sequences.

FIG. 2A-2B. Radioreceptor binding assays using HKrk-B7 cells and ¹²⁵I-labeled rat [Nle^{8,21}, Tyr³⁴]PTH(1-34)amide (FIG. 2A) or [Tyr³⁶]PTHrP(1-36)amide (FIG. 2B). Binding of either radioligand was inhibited by increasing concentrations of TIP(1-39) (⋄), TIP(3-39) ( [symbol] ), TIP(9-39) ( [symbol] ), PTH(1-34) (▪), PTHrP(1-36) (▾), or PTHrP(1-20)/TIP(23-39) ( [symbol] ). Data are expressed as % of maximal specific binding and represent the results (mean±SE) of at least three independent experiments.

FIG. 3A-3B. Ligand-stimulated cAMP accumulation in HKrk-B7 cells stably expressing the recombinant human PTH1R (FIG. 3A) or in human osteoblast-like, osteosarcoma cells (SaOS-2) expressing the endogenous PTH1R (FIG. 3B). Cells were stimulated with increasing concentrations of PTH(1-34) (▪), PTHrP(1-36) (▾), or PTHrP(1-20)/TIP(23-39) (Δ). Data are expressed as % of maximal cAMP accumulation and represent the results (mean±SE) of at least three independent experiments.

FIG. 4A-4F. Inhibition of agonist-stimulated cAMP accumulation in HKrk-B7 cells (FIG. 4A-4C) or SaOS-2 cells (FIG. 4D-4F). Cells were stimulated with approximately half-maximal concentrations of either PTH(1-34), PTHrP(1-36), or PTHrP(1-20)/TIP(23-39) in the absence or presence of increasing concentrations of TIP(1-39) (⋄), TIP(9-39) ( [symbol] ), or PTHrP(7-36) (●); agonist concentrations were 1 nM for HKrk-B7 cells, and 0.15-0.3 nM for SaOS-2 cells. Data are expressed as % of half-maximal cAMP accumulation and represent the results (mean±SE) of at least three independent experiments.

FIG. 5. Binding of chimeric TIP polypeptides to the hPTH-1 receptor in KRK-B7 cells. The peptides indicated in the key were evaluated at varying doses for their capacity to inhibit the binding of ¹²⁵I-bPTH(3-34) tracer.

FIG. 6. Effect of chimeric TIP analogs and control peptides on cAMP formation in HKRK-B7 cells. hPTH1 receptor (B7 cells).

FIG. 7. Effect of amino-terminally modified analogs of TIP on cAMP formation in HRKR-B7 cells. PTH(1-34) was used as a postive control; the cells are stably transfected with the human PTH-1 receptor and do not express the PTH-2 receptor.

FIG. 8. Inhibition of agonist-stimulated cAMP accumulation. Cells were stimulated with approximately half-maximal concentrations of either PTH(1-34), or TIP(1-39) in the presence of increasing concentrations of TIP(9-39) in P2R LLCPK1 cells. Data are expressed as % of half-maximal cAMP accumulation and represent the results (mean±SE) of at least three independent experiments.

FIG. 9A-9B. Schematic representation of the known portions of the human (upper panel) and the mouse (lower panel) TIP39 gene (FIG. 9A). The names of the different exons are indicated; the sizes of exons (normal letters) and introns (italic letters) are given in bp; the approximate positions of the different PCR primers are shown; note that the positions of the universal AP1 and AP2 primers that were used for 5′ RACE are arbitrary. Splice donor/acceptor sites in the human and mouse gene are shown; exonic nucleotides are shown in capital letters; intronic nucleotides in lower case letters; splice site consensus nucleotides are in bold; the initiator ATG in exon 1 is underlined (FIG. 9B)[SEQ ID NOS: 22-29].

FIG. 10. Nucleotide sequence of the human TIP39 gene [SEQ ID NO:30]. Nucleotides found in the mature mRNA are capitalized, nucleotides in flanking intervening DNA sequences are in lower case. Because of uncertainty about the start site of transcription and the exact length of exon U1, the first nucleotide of the coding region is designated nucleotide +1. Splice donor and acceptor sites are underlined; a putative polyadenylation signal is shown in bold underlined lower case letters. Partial exon U1 sequence information (deduced from mouse TIP39) is in dark gray. Coding nucleotides are shaded in light gray. The amino acid sequence of the human precursor TIP39 is indicated below nucleotides. The secreted peptide sequence is boxed.

FIG. 11A-11B. Amino acid sequence alignment for the human and murine TIP39 precursors (FIG. 11A)[SEQ ID NOS: 32,33]. Residues that are identical (dark shade) or similar (light shade) in human and mouse TIP39 are boxed, the black bar depicts the secreted peptide with the first residue denoted as “+1”. Kyte/Doolittle hydrophobicity plot of the deduced human TIP39 precursor (upper panel) and mouse TIP39 precursor (lower panel) peptide sequence (FIG. 11B). The thick black bar depicts the secreted peptide; the position of the first residue is denoted as “+1”. The ordinate indicates relative hydrophobicity, with more positive values corresponding to increased hydrophobicity.

FIG. 12. Comparison of the gene structure for human TIP39, human PTH and human PTHrP. Boxed areas are exons and their names are shown underneath (since the start of exon U1 of the TIP39 gene is unknown, the box is open on the left site), white boxes denote presequences, black boxes denote prosequences (for TIP39 presumed), gray stippled boxes denote the mature sequences; noncoding regions are shown as striped boxes. The small striped boxes preceding the white boxes denote untranslated exonic sequences (4 bp for TIP39; 5 bp for PTH; 22 bp for PTHrP). The positions of the initiator methionine based on the secreted peptide are noted above the graphs; the positions where pro-sequences are interrupted by an intron are noted above the graph. +1 denotes the relative position of the beginning of the secreted peptide.

FIG. 13. Phylogenetic analysis indicating the evolutionary relationship among precursor proteins of the TIP39, PTH, PTHrP and secretin families of peptides. A Neighbor-Joining phylogenetic analysis using distance as the criteria is shown above (tree length=740, consistency index excluding uninformative residues=0.876, with 165 parsimony-informative characters). The bootstrap/jackknife values from 10,000 replicates indicate support of a given node where 95% is considered to be significant (Page, R., and Holmes, E. Molecular Evolution: a phylogenetic approach, Blackwell Science Ltd., Oxford, UK (1998); Felsenstein, J., and Kishino, H., Syst. Biol. 42:193-200 (1993)). A Maximum Parsimony analysis using parsimony as the criteria generated a similar phylogenetic relationship between PTH, PTHrP, TIP39, secretin, and GIP (tree length=746, consistency index excluding uninformative residues=0.846, with 165 parsimony-informative characters) (Swofford, D., et al., “Phylogenetic inference,” in Molecular Systematics, Hillis, D, et al., eds., Sinauer Associates, Inc., Sunderland, Mass. (1996), pp. 407-514; Page, R., and Holmes, E. Molecular Evolution: a phylogenetic approach, Blackwell Science Ltd., Oxford, UK (1998)). In addition, to the Neighbor-Joining and Maximum Parsimony phylogenetic analyses, Quartet puzzling using Maximum Parsimony criteria and Star-decomposition (tree length=739, consistency index excluding uninformative characters=0.855, with 165 parsimony-informative characters) support the hypothesis that PTH and PTHrP are sister groups, and that TIP39 is the sister group to this clade. PTH, PTHrP, and TIP39 thus form a superfamily, while secretin and VIP (not shown) appears to be a sister group to this larger superfamily (Accession numbers: PTH (cat, AF309967; chick, M36522; cow, J00024; dog, U15662; horse, AF134233; human, NM_{—}000315; macaque, AF130257; mouse, NM_{—}020623; pig, X05722; and rat, NM_{—}017044); PTHrP (chick, X52131; human, J03580; mouse, M60056; rabbit, AF219973; rat, NM_{—}012636; sheep, AF327654; fugu, AJ249391; sparus, AF197904); VIP (chick, U09350; mouse AK018599; human XM_{—}004381); secretin (mouse, X73580; pig, M31496; human, XM_{—}012014); and human GIP (NM_{—}004123))

FIG. 14A-14B. Ligand-stimulated cAMP accumulation in hPR2-20 LLCPK₁ cells stably expressing the recombinant human PTH2 receptor (FIG. 14A). Cells were stimulated with increasing concentrations of human TIP-(1-39) (●) or mouse TIP-(1-39) (▴). Data are expressed as picomoles per well and represent the results (mean±SEM) of two independent experiments; basal cAMP accumulation was 0.23 pmol/well. Inhibition of agonist stimulated cAMP accumulation in hPR2-20 LLCPK₁ cells (FIG. 14B). Cells were stimulated with approximately half-maximal concentrations of human TIP-(1-39) (●) or PTH-(1-34) (▪) in the absence or presence of increasing concentrations of TIP-(9-39). Data are expressed as percentage of half-maximal cAMP accumulation and represent the results (mean±SEM) of two independent experiments.

FIG. 15. Northern blot analysis of poly-A⁺0 RNA derived from several different mouse tissues using a cDNA probe encoding mouse TIP39 (nucleotides 1 to 472; AY048587). Note that poly-A⁺ RNA from testis showed three hybridizing bands; a prominent mRNA of approximately 4.5 kb and two larger transcripts that hybridize less intensely (left panel; final wash: 0.1×SSC, 0.1% SDS, 50° C., exposure for 3 days at −80° C.). Poly-A⁺ RNA from liver, kidney, and possibly heart revealed a single weakly hybridizing transcript that is approximately 4.5 kb in size, while poly-A⁺ RNA from liver showed an additional hybridizing band of about 1.5 kb (arrows), and poly-A⁺ RNA from brain showed evidence for transcripts of about 1 kb and possibly 0.7 kb (arrows) (right panel; 3 weeks exposure).

FIG. 16A-16F. TIP39 transcripts detected by RNA in situ hybridization using sagital sections of an adult mouse brain. Sagital section (H&E staining), corresponding to section 163 (Sidman, R. L., et al., Atlas of the Mouse Brain and Spinal Cord, Harvard University Press, Cambridge, Mass. (1971)). The arrow depicts nucleus subparafascicularis thalami (FIG. 16A). Close-up (approx. ×2) of the same section in dark field (FIG. 16B). Sagital section (H&E staining), roughly corresponding to section 143 (Sidman, R. L., et al., Atlas of the Mouse Brain and Spinal Cord, Harvard University Press, Cambridge, Mass. (1971)); left arrow: nucleus ruber, right arrow: nucleus centralis pontis (FIG. 16C). Close-up (approx. ×2) of the same section in dark field (FIG. 16D). Coronal section (H&E staining) (Bregma—2.92 mm (Franklin, K. B. J., and Paxinos, G., The Mouse Brain in Stereotaxic Coordinates, Academic Press, San Diego, Calif. (1997))), arrow depicting TIP39 expression in an area corresponding to nucleus subparafascicularis thalami (FIG. 16E). Close-up (approx. ×2) of the same section in dark field (FIG. 16F). The bar represents 1 mm for sagital (panel A, C) and 0.5 mm for coronal (panel E) sections.

FIG. 17. TIP39 transcripts detected by RNA in situ hybridization in seminiferous tubuli. Representative section through an adult mouse testis (left panel: H&E staining, right panel: dark field; original magnification, x200) showing strong TIP39 expression in segments, which correspond to different stages of the spermatogenic cycle.

### DETAILED DESCRIPTION OF THE INVENTION

In order to provide a clearer understanding of the specification and claims, the following definitions are provided.

### Abbreviations and Definitions

In order to provide a clear and consistent understanding of the specification and claims, including the scope to be given such terms, the following definitions are provided.

Reference to truncated forms of polypeptides, such as for example TIP3-39 refers to the TIP polypeptide beginning with amino acid number 3 from the N-terminal end of the molecule.

The one- and three-letter abbreviations for the various common nucleotide bases and amino acids are as recommended in Pure Appl. Chem. 31, 639-645 (1972) and 40, 277-290 (1974) and comply with 37 CFR § 1.822. The abbreviations represent L-amino acids unless otherwise designated as D- or D,L-. Certain amino acids, both natural and non-natural, are achiral, e.g. glycine. All peptide sequences are presented with the N-terminal amino acid on the left and the C-terminal amino acid on the right. In some variations of the invention, the amino acid sequences of the invention may use either D or D,L amino acids

Agonist: By “agonist” is intended a ligand capable of enhancing or potentiating a cellular response mediated by for example, the PTH-2 receptor or PTH/PTHrP receptor.

Antagonist: By “antagonist” is intended a ligand capable of inhibiting or attenuating a cellular response mediated by for example, the PTH/PTHrP or PTH2 receptor. Whether any candidate “agonist” or “antagonist” of the present invention can enhance or inhibit a cellular response can be determined using art-known protein ligand/receptor cellular response or binding assays, including those described elsewhere in this application.

Antibody—An “antibody” (interchangeably used in plural form) is an immunoglobulin molecule capable of specific binding to a target such as a polypeptide. As used herein, the term encompasses not only intact antibodies, but also fragments thereof, mutants thereof, fusion protein, humanized antibodies and any other modified configuration of the immunoglobulin molecule that comprises an antigen recognition site of the required specificity. Antibodies are made by methods readily known to those of skill in the art, such as for example, those found in Current Protocols in Molecular Biology ed. Ausubel et al., John Wiley Sons (1994).

Biological Activity of the Protein: This expression refers to the metabolic or physiologic function of compounds, for example, SEQ ID NO: 1 or derivatives thereof including similar activities or improved activities or those activities with decreased undesirable side-effects. Also included are antigenic and immunogenic activities of said compounds of, for example, SEQ ID NO: 1 or derivatives thereof.

Cloning vector: A plasmid or phage DNA or other DNA sequence which is able to replicate autonomously in a host cell, and which is characterized by one or a small number of restriction endonuclease recognition sites at which such DNA sequences may be cut in a determinable fashion without loss of an essential biological function of the vector, and into which a DNA fragment may be spliced in order to bring about its replication and cloning. The cloning vector may further contain a marker suitable for use in the identification of cells transformed with the cloning vector. Markers, for example, provide tetracycline resistance or ampicillin resistance.

Conservative Amino Acid Substitution: Conservative amino acid changes are of a minor nature and should not affect the activity of the polypeptide in question. Such as conservative amino acid substitutions do not significantly affect the folding or activity of the protein. Examples of conservative amino acid substitution can be found in Table 1.

Amino acids in the polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Amino acids in the polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904(1992) and de Vos et al. Science 255:306-312 (1992)).

Expression vector: A vector similar to a cloning vector but which is capable of enhancing the expression of a gene which has been cloned into it, after transformation into a host. The cloned gene is usually placed under the control of(i.e., operably linked to) certain control sequences such as promoter sequences. Promoter sequences may be either constitutive or inducible.

Fusion protein: By the term “fusion protein” is intended to mean a fused protein comprising compounds of for example, SEQ ID NO: 1 or derivatives thereof, either with or without a “selective cleavage site” linked at its N-terminus, which is in turn linked to an additional amino acid leader polypeptide sequence.

Fragment: A “fragment” of a molecule is meant to refer to any polypeptide or polynucleotide subset of the molecule of interest.

Functional Derivative: The term “derivatives” is intended to include “variants,” the “derivatives,” or “chemical derivatives” of the molecule. A “variant” of a molecule or derivative thereof is meant to refer to a molecule substantially similar to either the entire molecule, or a fragment thereof. An “analog” of a molecule or derivative thereof is meant to refer to a non-natural molecule substantially similar to for example, either the SEQ ID NO: 1 molecules or fragments thereof.

A molecule is said to be “substantially similar” to another molecule if the sequence of amino acids in both molecules is substantially the same, and if both molecules possess a similar biological activity. Thus, provided that two molecules possess a similar activity, they are considered variants, derivatives, or analogs as that term is used herein even if one of the molecules contains additional amino acid residues not found in the other, or if the sequence of amino acid residues is not identical.

As used herein, a molecule is said to be a “chemical derivative” of another molecule when it contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Examples of moieties capable of mediating such effects are disclosed in Remington's Pharmaceutical Sciences (1980) and will be apparent to those of ordinary skill in the art.

Gene Therapy: A means of therapy directed to altering the normal pattern of gene expression of an organism. Generally, a recombinant polynucleotide is introduced into cells or tissues of the organism to effect a change in gene expression.

Host Animal: Transgenic animals, all of whose germ and somatic cells contain the DNA construct of the invention. Such transgenic animals are in general vertebrates. Preferred host animals are mammals such as non-human primates, mice, sheep, pigs, cattle, goats, guinea pigs, rodents, e.g. rats, and the like. The term Host Animal also includes animals in all stages of development, including embryonic and fetal stages.

Homologous/Nonhomologous: Two nucleic acid molecules are considered to be “homologous” if their nucleotide sequences share a similarity of greater than 40%, as determined by HASH-coding algorithms (Wilber, W. J. and Lipman, D. J., Proc. Natl. Acad. Sci. 80:726-730 (1983)). Two nucleic acid molecules are considered to be “nonhomologous” if their nucleotide sequences share a similarity of less than 40%.

Isolated: A term meaning altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated for purposes of the present invention. Also intended as an “isolated polypeptide” or an “isolated polynucleotide” are polypeptides or polynucleotides that have been purified, partially or substantially, from a recombinant host cell or from a native source. For example, a recombinantly produced version of compounds of SEQ ID NO:1 and derivatives thereof can be substantially purified by the one-step method described in Smith and Johnson, Gene 67:31-40 (1988).

By “isolated” is meant that the DNA is free of the coding sequences of those genes that, in the naturally-occurring genome of the organism (if any) from which the DNA of the invention is derived, immediately flank the gene encoding the DNA of the invention. The isolated DNA may be single-stranded or double-stranded, and may be genomic DNA, cDNA, recombinant hybrid DNA, or synthetic DNA. It may be identical to a native DNA sequence encoding compounds of for example, SEQ ID NO:1 and derivatives thereof, or may differ from such sequence by the deletion, addition, or substitution of one or more nucleotides. Single-stranded DNAs of the invention are generally at least 8 nucleotides long, (preferably at least 18 nucleotides long, and more preferably at least 30 nucleotides long) ranging up to full length of the DNA molecule encoding compounds of for example, SEQ ID NO:1 and derivatives thereof; they preferably are detectably labeled for use as hybridization probes, and may be antisense.

Isolated or purified as it refers to preparations made from biological cells or hosts should be understood to mean any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange change chromatography, affinity chromatography, density gradient centrifugation and electrophoresis.

A preparation of DNA or protein that is “pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

While many embodiments of the claimed invention use isolated or purified polynucleotides or polypeptides, this need not always be the case. For example, a recombinant host cell expressing the novel receptors of the invention may be used in screening assays to identify PTH agonists without being further isolating the expressed receptor proteins.

High Stringency: By “high stringency” is meant, for example, conditions such as those described for the isolation of cDNA (also see Current Protocols in Molecular Biology, John Wiley & Sons, New York (1989), hereby incorporated, by reference). The DNA of the invention may be incorporated into a vector which may be provided as a purified preparation (e.g., a vector separated from the mixture of vectors which make up a library) containing a DNA sequence encoding a peptide of the invention (e.g. compounds of SEQ ID NO:1 and derivatives thereof) and a cell or essentially homogenous population of cells (e.g., prokaryotic cells, or eukaryotic cells such as mammalian cells) which contain the vector (or the isolated DNA described above). The invention is also drawn to nucleic acid sequences that bind to DNA sequences encoding polypeptides of the invention under high stringency conditions, such conditions being well known to those of skill in the art.

Identity: This term refers to a measure of the identity of nucleotide sequences or amino acid sequences. In general, the sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide. As used herein, the term at least 85% identical to refers to percent identities from 85 to 99.99 relative to the reference polypeptides or polynucleotides. Identity at a level of 85% or more is indicative of the fact that, assuming for exemplification purposes a test and reference polynucleotide length of 100 nucleotides, that no more than 15% (i.e., 15 out of 100) of the nucleotides in the test polynucleotides differ from that of the reference polynucleotide. Such differences may be represented as point mutations randomly distributed over the entire length of the sequence of the invention or they may be clustered in one or more locations. Differences are defined as amino acid or nucleotide substitutions or deletions.

As a practical matter, whether any particular nucleic acid molecule is at least 95%, 96%, 97%, 98% or 99% identical to the nucleotide sequences of the invention can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed. In this regard, it is possible to obtain sequences related to the specific sequences of the invention, such as for example SEQ. ID NO:1 or a nucleic acid encoding SEQ ID NO:1 by screening a genomic database screening, once the percent identity or homology of interest is determined.

One aspect of the present application is directed to nucleic acid molecules at least 95%, 96%, 97%, 98% or 99% identical to the nucleic acid sequence encoding the polypeptides of the invention.

Leader Sequence: By the term “leader sequence” is intended a polynucleotide sequence linked to for example, DNA encoding compounds of SEQ ID NO: 1, and expressed in host cells as a fusion protein fused to the selective cleavage site and compounds of SEQ ID NO: 1. The term “leader polypeptide” describes the expressed form of the “leader sequence” as obtained in the fusion protein.

The fusion protein, which is often insoluble and found in inclusion bodies when it is overexpressed, is purified from other bacterial protein by methods well known in the art. In a preferred embodiment, the insoluble fusion protein is centrifuged and washed after cell lysis, and resolubilized with guanidine-HCl. It can remain soluble after removal of the denaturant by dialysis. (For purification of refractile proteins, see Jones, U.S. Pat. No. 4,512,922; Olson, U.S. Pat. No. 4,518,526; and Builder et al., U.S. Pat. Nos. 4,511,502 and 4,620,948).

The recombinantly produced compounds of, for example, SEQ ID NO: 1 or derivatives thereof can be purified to be substantially free of natural contaminants from the solubilized fusion protein through the use of any of a variety of methodologies. As used herein, a compound is said to be “substantially free of natural contaminants” if it has been substantially purified from materials with which it is found following expression in bacterial or eukaryotic host cells. Compounds of SEQ ID NO: 1 or derivatives thereof may be purified through application of standard chromatographic separation technology.

Alternatively, the peptide may be purified using immuno-affinity chromatography (Rotman, A. et al., Biochim. Biophys. Acta 641:114-121 (1981); Sairam, M. R. J. Chromatog 215:143-152 (1981); Nielsen, L. S. et al., Biochemistry 21:6410-6415 (1982); Vockley, J. et al., Biochem. J. 217:535-542 (1984); Paucha, E. et al., J. Virol. 51:670-681 (1984); and Chong, P. et al., J. Virol. Meth. 10:261-268 (1985)).

After partial or substantial purification, the fusion protein is treated enzymatically with the enzyme corresponding to the cleavage site. Alternatively, the fusion protein in its more impure state, even in refractile form, can be treated with the enzyme. If needed, the resulting mature compounds of for example, SEQ ID NO: 1 or derivatives thereof, can be further purified. Conditions for enzymatic treatment are known to those of skill in the art.

Polynucleotide: This term generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

Polypeptide: This term refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in the research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins-Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Methods in Enzymol. 182:626-646 (1990) and Rattan et al., “Protein Synthesis: Posttranslational Modifications and Aging”, Ann NY Acad Sci 663:48-62 (1992). The polypeptides of the invention have a free amino group at the N-terminus and a carboxy-amid at the C-terminus.

Promoter: A DNA sequence generally described as the 5′ region of a gene, located proximal to the start codon. The transcription of an adjacent gene(s) is initiated at the promoter region. If a promoter is an inducible promoter, then the rate of transcription increases in response to an inducing agent. In contrast, the rate of transcription is not regulated by an inducing agent if the promoter is a constitutive promoter. Examples of promoters include the CMV promoter (InVitrogen, San Diego, Calif.), the SV40, MMTV, and hMTIIa promoters (U.S. Pat. No. 5,457,034), the HSV-14/5 promoter (U.S. Pat. No. 5,501,979), and the early intermediate HCMV promoter (WO92/17581). Also, tissue-specific enhancer elements may be employed. Additionally, such promoters may include tissue and cell-specific promoters of an organism.

Recombinant Host: According to the invention, a recombinant host may be any prokaryotic or eukaryotic host cell which contains the desired cloned genes on an expression vector or cloning vector. This term is also meant to include those prokaryotic or eukaryotic cells that have been genetically engineered to contain the desired gene(s) in the chromosome or genome of that organism. For examples of such hosts, see Sambrook et al., Molecular Cloning: A Laboratory Manual, Second Edition, Cold Spring Harbor Laboratory, Cold Spring Harbor, N.Y. (1989). Preferred recombinant hosts are eukaryotic cells transformed with the DNA construct of the invention. More specifically, mammalian cells are preferred.

Selective cleavage site: The term “selective cleavage site” refers to an amino acid residue or residues which can be selectively cleaved with either chemicals or enzymes in a predictable manner. A selective enzyme cleavage site is an amino acid or a peptide sequence which is recognized and hydrolyzed by a proteolytic enzyme. Examples of such sites include, without limitation, trypsin or chymotrypsin cleavage sites.

Truncated TIP39 polypeptide: “Truncated TIP39 polypeptide” refers to a polypeptide having a sequence comprising less than the full complement of amino acids found in TIP39. Examples of a truncated TIP39 polypeptide include, inter alia, TIP8-39, TIP9-39, TIP10-39, TIP11-39 and TIP12-39.

Administration of the Polypeptides of the Invention

In general, the agonist polypeptides of this invention, or salts thereof, are administered in amounts between about 0.01 and 1 μg/kg body weight per day, preferably from about 0.07 to about 0.2 μg/kg body weight per day. For a 50 kg human female subject, the daily dose of active ingredient is from about 0.5 to about 50 μgs, preferably from about 3.5 to about 10 μs. In other mammals, such as horses, dogs, and cattle, higher doses may be required. The dosage for the antagonist polypeptides may need to be 100-1000 fold higher than that for an agonist.

The dosage may be delivered in a conventional pharmaceutical composition by a single administration, by multiple applications, or via controlled release, as needed to achieve the most effective results, preferably one or more times daily by injection.

The selection of the exact dose and composition and the most appropriate delivery regimen will be influenced by, inter alia, the pharmacological properties of the selected polypeptide, the nature and severity of the condition being treated, and the physical condition and mental acuity of the recipient.

Representative delivery regimens include oral, parenteral (including subcutaneous, intramuscular and intravenous), rectal, buccal (including sublingual), transdermal, inhalation and intranasal.

Pharmaceutically acceptable salts retain the desired biological activity of the parent polypeptide without toxic side effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, naphthalenesulfonic acids, naphthalene disulfonic acids, polygalacturonic acid and the like; (b) base addition salts formed with polyvalent metal cations such as zinc, calcium, bismuth, barium, magnesium, aluminum, copper, cobalt, nickel, cadmium, and the like; or with an organic cation formed from N,N′-dibenzylethylenediamine or ethylenediamine; or (c) combinations of (a) and (b), e.g., a zinc tannate salt and the like.

A further aspect of the present invention relates to pharmaceutical compositions comprising as an active ingredient a polypeptide of the present invention, or pharmaceutically acceptable salt thereof, in admixture with a pharmaceutically acceptable, non-toxic carrier. As mentioned above, such compositions may be prepared for parenteral (subcutaneous, intramuscular or intravenous) administration, particularly in the form of liquid solutions or suspensions; for oral or buccal administration, particularly in the form of tablets or capsules; for intranasal administration, particularly in the form of powders, nasal drops or aerosols; and for rectal or transdermal administration.

The compositions may conveniently be administered in unit dosage form and may be prepared by any of the methods well-known in the pharmaceutical art, for example as described in Remington's Pharmaceutical Sciences, 17th ed., Mack Publishing Company, Easton, Pa., (1985), incorporated herein by reference. Formulations for parenteral administration may contain as excipients sterile water or saline, alkylene glycols such as propylene glycol, polyalkylene glycols such as polyethylene glycol, oils of vegetable origin, hydrogenated naphthalenes and the like. For oral administration, the formulation can be enhanced by the addition of bile salts or acylcarnitines. Formulations for nasal administration may be solid and may contain excipients, for example, lactose or dextran, or may be aqueous or oily solutions for use in the form of nasal drops or metered spray. Example of nasal administration of polypeptides can be found in U.S. Pat. No. 6,004,574. For buccal administration typical excipients include sugars, calcium stearate, magnesium stearate, pregelatinated starch, and the like.

When formulated for nasal administration, the absorption across the nasal mucous membrane may be enhanced by surfactant acids, such as for example, glycocholic acid, cholic acid, taurocholic acid, ethocholic acid, deoxycholic acid, chenodeoxycholic acid, dehydrocholic acid, glycodeoxycholic acid, cyclodextrins and the like in an amount in the range between about 0.2 and 15 weight percent, preferably between about 0.5 and 4 weight percent, most preferably about 2 weight percent.

Delivery of the compounds of the present invention to the subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, osmotic pumps, vesicles, micelies, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

One form of controlled release formulation contains the polypeptide or its salt dispersed or encapsulated in a slowly degrading, non-toxic, non-antigenic polymer such as copoly(lactic/glycolic) acid, as described in of Kent, Lewis, Sanders, and Tice, U.S. Pat. No. 4,675,189, incorporated by reference herein. The compounds or, preferably, their relatively insoluble salts, may also be formulated in cholesterol or other lipid matrix pellets, or silastomer matrix implants. Additional slow release, depot implant or injectable formulations will be apparent to the skilled artisan. See, for example, Sustained and Controlled Release Drug Delivery Systems, J. R. Robinson ed., Marcel Dekker, Inc., New York, 1978, and R. W. Baker, Controlled Release of Biologically Active Agents, John Wiley & Sons, New York, 1987, incorporated by reference herein.

Vectors, Host Cells, and Recombinant Expression

The present invention also relates to host cell and vectors that comprise a polynucleotide of the present invention, i.e. polynucleotides that encode the polypeptides of the invention, as well as the uses such vectors and host cells for treating (either in vivo or in vitro) conditions requiring agonist or antagonists of PTH receptors. Such polynucleotide sequences are easily designed by those skilled in the art using the truncated peptide sequences provided herein. Host cells which are genetically engineered with vectors of the invention may be used in the production of truncated or chimeric TIP polypeptides of the invention by recombinant techniques. Cell-free translation systems can also be employed to produce such proteins using RNAs derived from the DNA constructs of present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., Molecular Cloning: A Laboratory Manual (supra).

RNA vectors may also be utilized for the expression of the nucleic acids encoding compounds or derivatives thereof disclosed in this invention. These vectors are based on positive or negative strand RNA viruses that naturally replicate in a wide variety of eukaryotic cells (Bredenbeek, P. J. & Rice, C. M., Virology 3:297-310, 1992). Unlike retroviruses, these viruses lack an intermediate DNA life-cycle phase, existing entirely in RNA form. For example, alpha viruses are used as expression vectors for foreign proteins because they can be utilized in a broad range of host cells and provide a high level of expression; examples of viruses of this type include the Sindbis virus and Semliki Forest virus (Schlesinger, S., TIBTECH 11: 18-22, 1993; Frolov, I., et al., Proc. Natl. Acad. Sci. USA 93:11371-11377, 1996). As exemplified by Invitrogen's Sinbis expression system, the investigator may conveniently maintain the recombinant molecule in DNA form (pSinrep5 plasmid) in the laboratory, but propagation in RNA form is feasible as well. In the host cell used for expression, the vector containing the gene of interest exists completely in RNA form and may be continuously propagated in that state if desired.

For secretion of the translated protein into the lumen of the endoplasmic reticulum, into the periplasmic space or into the extracellular environment appropriate secretion signals may be incorporated into the desired polypeptide. These signals may be endogenous to the polypeptide or they may be heterologous signals.

The expression of a DNA sequence requires that the DNA sequence be “operably linked” to DNA sequences which contain transcriptional and translational regulatory information. An operable linkage is a linkage in which the control or regulatory DNA sequences and the DNA sequence sought to be expressed are connected in such a way as to permit gene expression. The precise nature of the “control regions” needed for gene expression may vary from organism to organism, but shall in general include a promoter region which, in prokaryotic cells, contains both the promoter (which directs the initiation of RNA transcription) as well as DNA sequences which, when transcribed into RNA, will signal the initiation of protein synthesis. Regulatory regions in eukaryotic cells will in general include a promoter region sufficient to direct the initiation of RNA synthesis.

Two DNA sequences are said to be operably linked if the nature of the linkage between the two DNA sequences does not (1) result in the introduction of a frameshift mutation, (2) interfere with the ability of the promoter region sequence to direct the transcription of the fusion protein-encoding sequence or (3) interfere with the ability of the fusion protein-encoding sequence to be transcribed by the promoter region sequence. Thus, a promoter region would be operably linked to a DNA sequence if the promoter were capable of transcribing that DNA sequence.

The joining of various DNA fragments, to produce the expression vectors of this invention is performed in accordance with conventional techniques, employing blunt-ended or staggered-ended termini for ligation, restriction enzyme digestion to provide appropriate termini, filling in of cohesive ends as appropriate, alkali and phosphatase treatment to avoid undesirable joining, and ligation with appropriate ligates. In the case of a fusion protein, the genetic construct encodes an inducible promoter which is operably linked to the 5′ gene sequence of the fusion protein to allow efficient expression of the fusion protein.

To express compounds of the invention or a derivative thereof in a prokaryotic cell (such as, for example, E. coli, B. subtilis, Pseudomonas, Streptomyces, etc.), it is necessary to operably link the SEQ ID NO: 1-encoding DNA sequence to a functional prokaryotic promoter. Such promoters may be either constitutive or, more preferably, regulatable (i.e., inducible or derepressible). Examples of constitutive promoters include the int promoter of bacteriophage λ, the bla promoter of the β-lactamase gene of pBR322, and the CAT promoter of the chloramphenicol acetyl transferase gene of pBR325, etc. Examples of inducible prokaryotic promoters include the major right and left promoters of bacteriophage λ, (PL and PR), the trp, recA lacZ lacI and gal promoters of E. coli, the α-amylase (Ulmanen, I. et al., J. Bacteriol. 162:176-182 (1985)), and the α-28-specific promoters of B. subtilis (Gilman, M. Z. et al., Gene 32:11-20 (1984)), the promoters of the bacteriophages of Bacillius (Gryczan, T. J., In: The Molecular Biology of the Bacilli, Academic Press, Inc., NY (1982)), and Streptomyces promoters (Ward, J. M. et al., Mol. Gen. Genet. 203:468-478 (1986)). Prokaryotic promoters are reviewed by Glick, B. R., J. Ind. Microbiol. 1:277-282 (1987); Cenatiempo, Y., Biochimie 68:505-516 (1986)); and Gottesman, S., Ann. Rev. Genet. 18:415-442 (1984)).

A prokaryotic promoter that may be used for this invention is the E. coli trp promoter, which is inducible with indole acrylic acid. If expression is desired in a eukaryotic cell, such as yeast, fungi, mammalian cells, or plant cells, then it is necessary to employ a promoter capable of directing transcription in such a eukaryotic host. Preferred eukaryotic promoters include the promoter of the mouse metallothionein I gene (Hamer, D. et al., J. Mol. Appl. Gen. 1:273-288 (1982)); the TK promoter of Herpes virus (McKnight, S., Cell 31:355-365 (1982)); the SV40 early promoter (Benoist, C., et al., Nature (London) 290:304-310 (1981)); and the yeast ga14 gene promoter (Johnston, S. A., et al., Proc. Natl. Acad. Sci. (USA) 79:6971-6975 (1982); Silver, P. A., et al., Proc. Natl. Acad. Sci. (USA) 81:5951-5955 (1984)).

Preferably, the introduced gene sequence will be incorporated into a plasmid or viral vector capable of autonomous replication in the recipient host. Any of a wide variety of vectors may employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColE1, pSC101, pACYC 184, πVX. Such plasmids are, for example, disclosed by Maniatis, T., et al., In: Molecular Cloning, A Laboratory Manual, Cold Spring Harbor Press, Cold Spring Harbor, N.Y. (1982)). Preferred plasmid expression vectors include the pGFP-1 plasmid described in Gardella et al., J. Biol. Chem. 265:15854-15859 (1989), or a modified plasmid based upon one of the pET vectors described by Studier and Dunn, Methods in Enzymology 185: 60-89 (1990). Bacillus plasmids include pC194, pC221, pT127, etc. Such plasmids are disclosed by Gryczan, T. In: The Molecular Biology of the Bacilli, Academic Press, NY pp. 307-329 (1982). Suitable Streptomyces plasmids include pIJIOI (Kendall, K. J. et al., J. Bacteriol. 169:4177-4183 (1987)), and Streptomyces bacteriophages such as φC31 (Chater, K. F. et al., In: Sixth International Symposium on Actinomycetales Biology, Akademiai Kaido, Budapest, Hungary, pp.45-54 (1986)). Pseudomonas plasmids are reviewed by John, J. F. et al., Rev. Infect. Dis. 8:693-704 (1986)), and Izaki, K., Jon. J. Bacteriol. 33:729-742 (1978)).

Preferred eukaryotic expression vectors include, without limitation, BPV, vaccinia, 2-micron circle etc. Such expression vectors are well known in the art (Botstein, D., et al., Miami Wntr. Symp. 19:265-274 (1982); Broach, J. R., In: The Molecular Biology of the Yeast Saccharomyces: Life Cycle and Inheritance, Cold Spring Harbor Laboratory, Cold Spring Harbor, N.Y. pp. 445-470 (1981); Broach, J. R., Cell 28:203-204 (1982); Bollon, D. P., et al., J. Clin. Hematol. Oncol. 10:39-48 (1980); Maniatis, T., In: Cell Biology: A Comprehensive Treatise, Vol. 3, Gene Expression, Academic Press, NY, pp. 563-608 (1980)).

In addition to microorganisms, cultures of cells derived from multicellular organisms may also be used as hosts. In principle, any such cell culture is workable, whether from vertebrate or invertebrate cellular sources. Interest, however, has been greater with cells from vertebrate sources. Examples of useful vertebrate host cell lines are VERO and HeLa cells, Chinese hamster ovary (CHO) cell lines, W138, BHK, COS-7, and MDCK cell lines. Expression vectors for such cells ordinarily include (if necessary) an origin of replication, a promoter located in front of or upstream to the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, Simian Virus 40 (SV40) and cytomegalovirus. The early and late promoters of SV40 virus are particularly useful because both are obtained easily from the virus as a fragment which also contains the SV40 viral origin of replication (Fiers et al., Nature 273:113 (1978)).

An origin of replication may be provided either by construction of the vector to include an exogenous origin, such as may be derived from SV40 or other viral (e.g. Polyoma, Adeno, VSV, BPV) source or may be provided by the host cell chromosomal replication mechanism. If the vector is integrated into the host cell chromosome, the latter is often sufficient.

If cells without formidable cell membrane barriers are used as host cells, transfection is carried out by the calcium phosphate precipitation method as described by Graham and Van der Erb, Virology 52:546 (1978). However, other methods for introducing DNA into cells, such as by nuclear injection or by protoplast fusion may also be used. In the case of gene therapy, the direct naked plasmid or viral DNA injection method, with or without transfection-facilitating agents such as, without limitation, liposomes, provides an alternative approach to the current methods of in vivo or in vitro transfection of mammalian cells. If prokaryotic cells or cells which contain substantial cell wall constructions are used, the preferred method of transfection is calcium treatment, using calcium chloride as described in Cohen et al., Proc. Natl. Acad. Sci. USA 69:2110 (1972).

Gene Therapy

A patient (human or non-human) suffering from symptoms of a condition characterized by 1) requiring antagonism of PTH1R or PTH2R, 2) increrases in calcium resulting from excess PTH or PTHrP, 3) decreases in bone mass, or 4) an abonormality related to the activated PTH2R may be treated by gene therapy. By undertaking this approach, there should be an attenuation of the symptoms. Gene therapy has proven effective or has been considered to have promise in the treatment of certain forms of human hemophilia (Bontempo, F. A., et al., Blood 69:1721-1724 (1987); Palmer, T. D., et al., Blood 73:438-445 (1989); Axelrod, J. H., et al., Proc. Natl. Acad. Sci. USA 87:5173-5177 (1990); Armentano, D., et al., Proc. Natl. Acad. Sci. USA 87:6141-6145 (1990)), as well as in the treatment of other mammalian diseases such as cystic fibrosis (Drumm, M. L., et al., Cell 62:1227-1233 (1990); Gregory, R. J., et al., Nature 347:358-363 (1990); Rich, D. P., et al., Nature 347:358-363 (1990)), Gaucher disease (Sorge, J., et al., Proc. Natl. Acad. Sci. USA 84:906-909 (1987); Fink, J. K., et al., Proc. Natl. Acad. Sci. USA 87:2334-2338 (1990)), muscular dystrophy (Partridge, T. A., et al., Nature 337:176-179 (1989); Law, P. K., et al., Lancet 336:114-115 (1990); Morgan, J. E., et al., J. Cell Biol. 111:2437-2449(1990)), and metastatic melanoma (Rosenberg, S. A., et al., Science 233:1318-1321 (1986); Rosenberg, S. A., et al., N. Eng. J. Med. 319:1676-1680 (1988); Rosenberg, S. A., et al., N. Eng. J. Med. 323:570-578 (1990)).

In a preferred approach, a polynucleotide having the nucleotide sequence of a polypeptide of the invention may be incorporated into a vector suitable for introducing the nucleic acid molecule into cells of the mammal to be treated, to form a transfection vector.

A variety of vectors have been developed for gene delivery and possible gene therapy. Suitable vectors for this purpose include retroviruses, adenoviruses and adeno-associated viruses (AAV). Alternatively, the nucleic acid molecules of the invention may be complexed into a molecular conjugate with a virus (e.g., an adenovirus) or with viral components (e.g., viral capsid proteins). The vectors derive from herpes simplex virus type 1 (HSV-1), adenovirus, adeno-associated virus (AAV) and retrovirus constructs (for review see Friedmann, T., Trends Genet. 10:210-214 (1994); Jolly, D., Cancer Gene Therapy 1 (1994); Mulligan, R. C., Science 260:926-932 (1993); Smith, F. et al., Rest. Neurol. Neurosci. 8:21-34 (1995)). Vectors based on HSV-1, including both recombinant virus vectors and amplicon vectors, as well as adenovirus vectors can assume an extrachromosomal state in the cell nucleus and mediate limited, long term gene expression in postmitotic cells, but not in mitotic cells. HSV-1 amplicon vectors can be grown to relatively high titers (10⁷ transducing units/ml) and have the capacity to accommodate large fragments of foreign DNA (at least 15 kb, with 10 concatemeric copies per virion). AAV vectors (rAAV), available in comparable titers to amplicon vectors, can deliver genes (<4.5 kb) to postmitotic, as well as mitotic cells in combination with adenovirus or herpes virus as helper virus. Long term transgene expression is achieved by replication and formation of “episomal” elements and/or through integration into the host cell genome at random or specific sites (for review see Samulski, R. J., Current Opinion in Genetics and Development 3:74-80 (1993); Muzyczka, N., Curr. Top. Microbiol. Immunol. 158:97-129 (1992)). HSV, adenovirus and rAAV vectors are all packaged in stable particles. Retrovirus vectors can accommodate 7-8 kb of foreign DNA and integrate into the host cell genome, but only in mitotic cells, and particles are relatively unstable with low titers. Recent studies have demonstrated that elements from different viruses can be combined to increase the delivery capacity of vectors. For example, incorporation of elements of the HIV virion, including the matrix protein and integrase, into retrovirus vectors allows transgene cassettes to enter the nucleus of non-mitotic, as well as mitotic cells and potentially to integrate into the genome of these cells (Naldini, L. et al., Science 272:263-267 (1996)); and inclusion of the vesicular somatitis virus envelope glycoprotein (VSV-G) increases stability of retrovirus particles (Emi, N. et al., J. Virol. 65:1202-1207 (1991)).

HSV-1 is a double-stranded DNA virus which is replicated and transcribed in the nucleus of the cell. HSV-1 has both a lytic and a latent cycle. HSV-1 has a wide host range, and infects many cell types in mammals and birds (including chicken, rat, mice monkey, and human) Spear et al., DNA Tumor Viruses, J. Tooze, Ed. (Cold Spring Harbor Laboratory, Cold Spring Harbor, N.Y., 1981), pp. 615-746. HSV-1 can lytically infect a wide variety of cells including neurons, fibroblasts and macrophages. In addition, HSV-1 infects post-mitotic neurons in adult animals and can be maintained indefinitely in a latent state (Stevens, Current Topics in Microbiology and Immunology 70: 31 (1975)). Latent HSV-1 is capable of expressing genes.

AAV also has a broad host range and most human cells are thought to be infectable. The host range for integration is believed to be equally broad. AAV is a single stranded DNA parvovirus endogenous to the human population, making it a suitable gene therapy vector candidate. AAV is not associated with any disease, therefore making it safe for gene transfer applications (Cukor et al., The Parvoviruses, Ed. K. I. Berns, Plenum, N.Y., (1984) pp. 33-36; Ostrove et al., Virology 113: 521 (1981)). AAV integrates into the host genome upon infection so that transgenes can be expressed indefinitely (Kotin et al., Proc. Natl. Acad. Sci. USA 87: 221 (1990); Samulski et al., EMBO J. 10: 3941 (1991)). Integration of AAV into the cellular genome is independent of cell replication which is particularly important since AAV can thus transfer genes into quiescent cells (Lebkowski et al., Mol. Cell. Biol. 8:3988 (1988)).

Both HSV and AAV can deliver genes to dividing and non-dividing cells. In general, HSV virions are considered more highly infectious that AAV virions, with a ratio of virus particles: infectious units in the range of 10 for HSV (Browne, H. et al., J. Virol. 70:4311-4316 (1996)) and up to thousands for AAV (Snyder, R. O. et al., In Current Protocols in Human Genetics, Eds. Dracopoli, N. et al., John Wiley and Sons: New York (1996), pp. 1-24), and both having a broad species range. Still, each virion has specific trophisms which will affect the efficiency of infection of specific cell types. The recent identification of a membrane receptor for HSV-1 which is a member of the tumor necrosis factor alpha family (Montgomery, R. I. et al., 21st Herpes Virus Workshop Abstract # 167 (1996)) indicates that the distribution of this receptor will affect the relative infectability of cells, albeit most mammalian cell types appear to be infectable with HSV-1. AAV also has a very wide host and cell type range. The cellular receptor for AAV is not known, but a 150 kDA glycoprotein has been described whose presence in cultured cells correlates with their ability to bind AAV (Mizukami, H. et al., Virology 217:124-130 (1996)).

Techniques for the formation of such vectors comprising a gene encoding an agonist or antagonist of the invention are well-known in the art, and are generally described in “Working Toward Human Gene Therapy,” Chapter 28 in Recombinant DNA, 2nd Ed., Watson, J. D. et al., eds., New York: Scientific American Books, pp. 567-581 (1992). In addition, general methods for construction of gene therapy vectors and the introduction thereof into affected animals for therapeutic purposes may be found in the above-referenced publications, the disclosures of which are specifically incorporated herein by reference in their entirety.

In one general method, vectors comprising polynucleotides encoding an antagonist or agonist of the invention are directly introduced into the cells or tissues of the affected individual, preferably by injection, inhalation, ingestion or introduction into a mucous membrane via solution; such an approach is generally referred to as “in vivo” gene therapy. Alternatively, cells or tissues, e.g., hematopoietic cells from bone marrow, may be removed from the affected animal and placed into culture according to methods that are well-known to one of ordinary skill in the art; the vectors comprising the polynucleotides may then be introduced into these cells or tissues by any of the methods described generally above for introducing isolated polynucleotides into a cell or tissue, and, after a sufficient amount of time to allow incorporation of the polynucleotides, the cells or tissues may then be re-inserted into the affected animal or a second animal in need of treatment. Since the introduction of the DNA of interest is performed outside of the body of the affected animal, this approach is generally referred to as “ex vivo” gene therapy.

For both in vivo and ex vivo gene therapy, the polynucleotides of the invention may alternatively be operatively linked to a regulatory DNA sequence, which may be a heterologous regulatory DNA sequence, to form a genetic construct as described above. This genetic construct may then be inserted into a vector, which is then directly introduced into the affected animal in an in vivo gene therapy approach, or into the cells or tissues of the affected animal in an ex vivo approach. In another preferred embodiment, the genetic construct may be introduced into the cells or tissues of the animal, either in vivo or ex vivo, in a molecular conjugate with a virus (e.g., an adenovirus) or viral components (e.g., viral capsid proteins).

The above approaches result in (a) homologous recombination between the nucleic acid molecule and the defective gene in the cells of the affected animal; (b) random insertion of the gene into the host cell genome; or (c) incorporation of the gene into the nucleus of the cells where it may exist as an extrachromosomal genetic element. General descriptions of such methods and approaches to gene therapy may be found, for example, in U.S. Pat. No. 5,578,461; WO 94/12650; and WO 93/09222.

Therapeutic Uses of the Invention or Derivatives Thereof

Some forms of hypercalcemia and hypocalcemia are related to the interaction between PTH or PTHrP and the PTH-1 receptors. Hypercalcemia is a condition in which there is an abnormal elevation in serum calcium level; it is often associated with other diseases, including hyperparathyroidism, osteoporosis, carcinomas of the breast, lung, kidney and prostate, epidermoid cancers of the head and neck and of the esophagus, multiple myeloma, and hypernephroma. Hypocalcemia, a condition in which the serum calcium level is abnormally low, may result from a deficiency of effective PTH, e.g., following thyroid surgery or congenital lack of parathyroid tissue.

A method of the invention treats hyperparathyroidism. Hyperparathyroidism is a condition due to an increase in the secretion of the parathyroids, causing generalized osteitis fibrosa cystica, elevated serum calcium, decreased serum phophorus and increased liberation of both calcium and phosphorous from bone. The sine qua non of primary hyperparthyroidism is hypercalcemia. Hypercalcemia, however, has many origins other than primary hyperparathyroidism including, for example, hypervitaminosis D, granulomatous disease, use of thiazide drugs and non-endocrine tumors.

Additionally, compounds of the invention or derivatives thereof are useful for the prevention and treatment of a variety of mammalian conditions manifested by loss of bone mass. In particular, for example, the chimeric polypeptide PTHrP(1-20)/TIP(23-39) [SEQ ID NO:8] of this invention is indicated for the prophylaxis and therapeutic treatment of osteoporosis and osteopenia in humans. Furthermore, the compounds of this invention are indicated for the prophylaxis and therapeutic treatment of other bone diseases. The compounds of this invention are indicated for the prophylaxis and therapeutic treatment of hypoparathyroidism. Finally, the compounds of this invention are indicated for use as agonists for fracture repair.

An example of a disease associated with bone loss is osteoporosis. Osteoporosis is a potentially crippling skeletal disease observed in a substantial portion of the senior adult population, in pregnant women and even in juveniles. The disease is marked by diminished bone mass, decreased bone mineral density (BMD), decreased bone strength and an increased risk of bone fracture. At present, there is no effective cure for osteoporosis, though estrogen, calcitonin and the bisphosphonates, etidronate and alendronate are used to treat the disease with varying levels of success through their action to decrease bone resorption. Since parathyroid hormone regulates blood calcium and phosphate levels, and has potent anabolic (bone-forming) effects on the skeleton, in animals (Shen, V., et al., Calcif. Tissue Int. 50:214-220 (1992); Whitefild, J. F., et al., Calcif. Tissue Int. 56:227-231 (1995) and Whitfield, J. F., et al., Calcif. Tissue Int. 60:26-29 (1997)) and humans (Slovik, D. M., et al., J Bone Miner. Res. 1:377-381 (1986); Dempster, D. W., et al., Endocr. Rev. 14:690-709 (1993) and Dempster, D. W., et al., Endocr. Rev. 15:261 (1994)) when administered intermittently, PTH, or PTH derivatives, are prime candidates for new and effective therapies for osteoporosis.

In general, agonist compounds or derivatives thereof of this invention, or salts thereof, are administered in amounts between about 0.01 and 1 μg/kg body weight per day, preferably from about 0.07 to about 0.2 μg/kg body weight per day. For a 50 kg human female subject, the daily dose of biologically active compounds of SEQ ID NO: 1 or derivatives thereof is from about 0.5 to about 50 μgs, preferably from about 3.5 to about 10 μgs. In other mammals, such as horses, dogs, and cattle, higher doses may be required. This dosage may be delivered in a conventional pharmaceutical composition by a single administration, by multiple applications, or via controlled release, as needed to achieve the most effective results, preferably one or more times daily by injection. For example, this dosage may be delivered in a conventional pharmaceutical composition by nasal insufflation.

Nucleic acids of the invention which encode polypeptides of the invention or derivatives thereof may be linked to a selected tissue-specific promoter and/or enhancer and the resultant hybrid gene introduced, by standard methods (e.g., as described by Leder et al., U.S. Pat. No. 4,736,866, herein incorporated by reference), into an animal embryo at an early developmental stage (e.g., the fertilized oocyte stage), to produce a transgenic animal which expresses elevated levels of the polypeptide of the invention or derivatives thereof in selected tissues (e.g., the osteocalcin promoter for bone). Such promoters are used to direct tissue-specific expression of compounds of SEQ ID NO: 1 or derivatives thereof in the transgenic animal.

In addition, any other amino-acid substitutions of a nature, which do not destroy the ability of the compounds of the invention to antagonize or agonize the PTH-1 or PTH-2 receptor (as determined by assays known to the skilled artisan and discussed below), are included in the scope of the present invention.

In accordance with one aspect of the invention, there is provided a method for treating a medical disorder that results from altered or excessive action of the PTH-1 or PTH-2 receptor, comprising administering to a patient a therapeutically effective amount of a polypeptide of the invention or a derivative thereof sufficient to inhibit activation of the PTH-1 or PTH-2 receptor of said patient.

In this embodiment, a patient who is suspected of having a disorder resulting from altered action of the PTH-1 or PTH-2 receptor may be treated using polypeptides of the invention or derivatives thereof of the invention which are a selective antagonists of the PTH-1 or PTH-2 receptor. Such antagonists include compounds of the invention or derivatives thereof of the invention which have been determined (by the assays described herein) to interfere with PTH-1 or PTH-2 receptor-mediated cell activation or other derivatives having similar properties.

To administer the antagonist, the appropriate compound of the invention or a derivative thereof is used in the manufacture of a medicament, generally by being formulated in an appropriate carrier or excipient such as, e.g., physiological saline, and preferably administered intravenously, intramuscularly, subcutaneously, orally, or intranasally, at a dosage that provides adequate inhibition of the PTH-1 or PTH-2 receptor. Typical dosage could be 1 ng to 10 mg of the peptide per kg body weight per day.

In accordance with yet a further aspect of the invention, there is provided a method for treating osteoporosis, comprising administering to a patient a therapeutically effective amount of the chimeric polypeptide of the invention or a derivative thereof, sufficient to activate the PTH-1 receptor of said patient. Similar dosages and administration as described above for the antagonist, may be used for administration of the agonist, e.g., for treatment of conditions such as osteoporosis, other metabolic bone disorders, and hypoparathyroidism and related disorders.

### EXAMPLE 1

### Truncated and Chimeric TIP39 Polypeptides

From the apparent, albeit limited structural homology within the carboxyl-terminal region of all three peptides, it appeared plausible that TIP(1-39) would be able to bind to the PTH1R without activating it. To test this hypothesis, TIP(1-39), several truncation mutants of this peptide, as well as several PTHrP/TIP chimeras were synthesized and their capacity to functionally interact with the PTH1R was assessed. The results reveal similarities and differences in the receptor interaction properties of TIP(1-39) and PTH or PTHrP.

Materials and Methods

Peptides

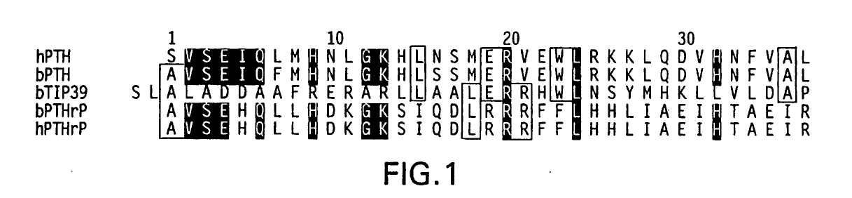

Peptides were synthesized by the Biopolymer Core Facility at Massachusetts General Hospital (Boston, Mass.) using Fmoc chemistry on Perkin-Elmer, Applied Biosystems synthesizers (model 430A or 431A). All peptides were purified to homogeneity by reversed-phase chromatography, and their sequence was confirmed by amino acid composition, amino acid sequence analysis, and mass spectroscopy. The following peptides were prepared (all TIP analogs are based on the bovine sequence (Usdin, T. B., et al., Nature Neuroscience 2:941-943 (1999)); all PTHrP analogs are based on the human sequence) (FIG. 1): TIP(1-39), TIP(3-39), TIP(9-39), TIP(19-39), and TIP(23-39), [Nle^{8,21}, Tyr³⁴] rat PTH(1-34)amide (rPTH), [Tyr³⁴] human PTH(1-34)amide (PTH(1-34)), [Tyr³⁶]PTHrP(1-36)amide (PTHrP(1-36)), PTHrP(1-20)amide (PTHrP(1-20)), PTHrP(1-6)/TIP(9-39), [Ile⁵]PTHrP(1-6)/TIP(9-39), [Ile⁷]TIP(1-39), PTHrP(1-20)/TIP(23-39), PTHrP(1-13)/TIP(16-39), PTHrP(1-9)/TIP(12-39) and [Leu¹¹, D-Trp¹², Trp²³, Tyr³⁶]PTHrP(7-36)amide (PTHrP(7-36)).

Polypeptides made be synthesized by any means known to those of skill in the art such as for example, those methods referred to in U.S. Pat. No. 5,693,616.

Cell Culture

DMEM, Trypsin/EDTA, and penicillin G/streptomycin and horse serum were from Gibco/BRL, Life Technologies, Gaithersburg, Md. LLC-PK, expressing the recombinant human PTH1R (HKrk-B7 cells) and SaOS-2 cells expressing the wild-type PTH1R endogenously were cultured in DMEM supplemented with 10% heat-inactivated fetal bovine serum, 100 U/ml penicillin, 100 μg/ml streptomycin, as previously described (Gardella, T. J., et al., J. Biol. Chem. 271:19888-19893 (1996); Fukayama, S., et al., Endocrinology 134:1851-1858 (1994)); both cell lines were maintained in a humidified atmosphere containing 95% air and 5% CO₂. After seeding, medium was replaced daily, until cells were used for radioligand binding or cAMP accumulation assays.

Radioreceptor Assays and Stimulation of cAMP Accumulation

Na¹²⁵I (specific activity: 2,000 Ci/mmol) was purchased from DuPont/NEN, Boston, Mass. Fetal bovine serum, 3-isobutyl-1-methyl-xanthine (IBMX), and BSA were from Sigma, St. Louis, Mo., and trifluoroacetic acid (TFA) was from Pierce, Rockford, Ill. Radiolabeled rPTH(1-34) and PTHrP(1-36) were prepared by the chloramine-T method, followed by HPLC purification using a 30-50% ACN/0.1% TFA gradient over 30 minutes; radioreceptor assays were performed in 24-well plates as previously described (Gardella, T. J., et al., J. Biol. Chem. 271:19888-19893 (1996); Fukayama, S. et al., Endocrinology 134:1851-1858 (1994)). In brief, each well (final volume 500 μl) contained binding buffer (BB; 50 mM Tris-HCl (pH 7.7), 100 mM NaCl, 5 mM KCl, 2 mM CaCl₂, 5% heat-inactivated horse serum, and 0.5% heat-inactivated fetal bovine serum), the ¹²⁵I-labeled PTH or PTHrP analog (100,000 to 200,000 cpm) was incubated in the absence or presence of increasing concentrations of unlabeled peptides. After 4 h at 16° C., buffer was completely removed, the cells were rinsed with cold BB and lysed with 1 M NaOH. The entire lysate was counted for γ-irradiation. Specific binding was determined after subtracting radioactivity bound in the presence of maximal concentrations of unlabeled competing peptide (10⁻⁶ M). Agonist-dependent stimulation of cAMP accumulation by HKrk-B7 and SaOS-2 cells (48-well plates; stimulation at room temperature for 45 minutes) and the subsequent measurement of cAMP by radioimmunoassay was performed as previously described (Gardella, T. J., et al., J. Biol. Chem. 271:19888-19893 (1996); Bergwitz, C., et al., Endocrinology 139:723-732 (1998)). Data were analyzed and graphically illustrated using the graph-pad prism software package.

Results

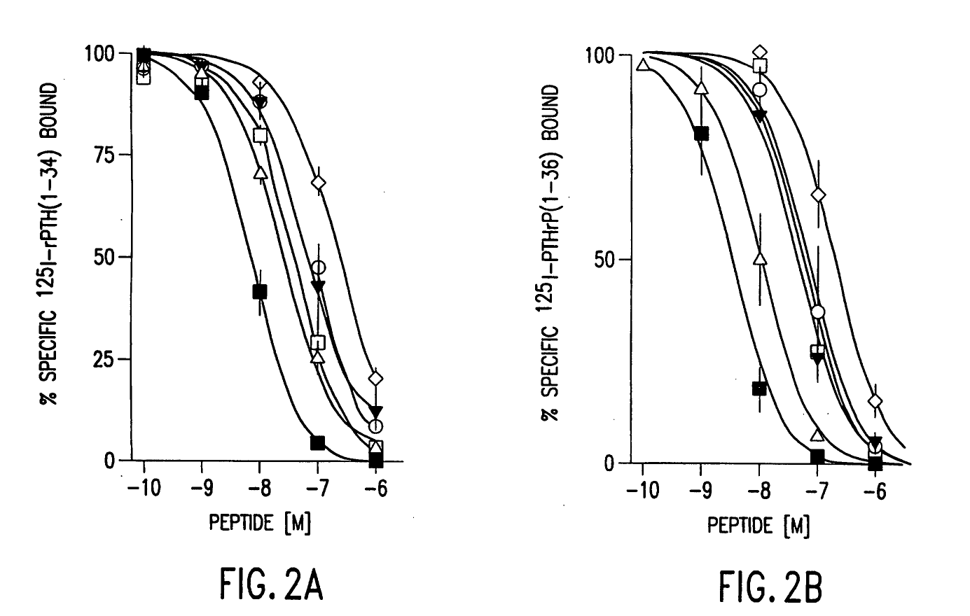

To determine whether TIP(1-39) or analogs thereof can interact with the PTH1R, the native peptide (TIP), several TIP analogs truncated at the amino-terminus (TIP(3-39), TIP(9-39), TIP(19-39), and TIP(23-39)) were synthesized, as well as several peptide chimeras. The binding properties of these peptides were evaluated in radioreceptor assays with HKrk-B7 cells, which express the PTH IR at high density (approximately 106 receptors/cell) (Carter, P. H., et al., Endocrinology 140:4972-4981 (1999)), using either radiolabeled rPTH(1-34) or PTHrP(1-36). Native TIP(1-39) bound to the PTH1R, although with considerably lower apparent affinity than did PTH(1-34) and PTHrP(1-36) (FIG. 2A-2B; Table 2). Removal of the first two or the first eight amino acid residues yielded TIP(3-39) and TIP(9-39), which exhibited improvements in apparent binding affinity of up to 6-fold relative to TIP(1-39). In fact, the apparent binding affinity of TIP(9-39) at the PTH1R was similar to that of the agonist PTHrP(1-36). TIP(19-39), TIP(23-39), as well as PTHrP(1-20) did not inhibit the binding of either radioligand (data not shown). In contrast, the chimera PTHrP(1-20)/TIP(23-39) exhibited high apparent binding affinity with IC₅₀ values of 31±8.2 nM when tested with radiolabeled rPTH(1-34), and 11±4.0 nM with radiolabeled PTHrP(1-36) (FIG. 2A-2B; Table 2). Thus, the binding affinity of PTHrP(1-20)/TIP(23-39) was only 3- to 4-fold weaker than that of PTH(1-34), yet 2- to 4-fold higher than that of PTHrP(1-36), and 8- to 19-fold higher than that of TIP(1-39). These findings suggested that the carboxyl-terminal region of TIP(1-39) can interact with the PTH1R, most likely at sites that overlap those used by PTH(1-34) and PTHrP(1-36).

Table 2: Peptide concentrations required for half-maximal inhibition of radioligand binding (IC₅₀):

TABLE 2

| | ¹²⁵I-rPTH(1-34) | ¹²⁵I-PTHrP(1-36) |
|---|---|---|
| Ligand | IC₅₀ (nM) | IC₅₀ (nM) |
| [Tyr³⁴]PTH(1-34) | 7.5 ± 1.7 | 3.4 ± 1.1 |
| [Tyr³⁶]PTHrP(1-36) | 78 ± 22 | 43 ± 7.0 |
| PTHrP(1-20)/TIP(23-39) | 31 ± 8.2 | 11 ± 4.0 |
| TIP(1-39) | 243 ± 52 | 210 ± 64 |
| TIP(3-39) | 96 ± 27 | 68 ± 19 |
| TIP(9-39) | 39 ± 10 | 44 ± 17 |

HKrk-B7 cells were incubated with ¹²⁵I-labeled rat [Nle^{8,21}, Tyr³⁴]PTH(1-34)amide or [Tyr³⁶]PTHrP(1-36)amide and increasing concentrations of PTH(1-34), PTHrP(1-36), or several analogs of TIP(1-39) (see FIG. 2). The calculated IC₅₀ values (mean±SE) are derived from at least three independent experiments.

The ability of TIP(1-39) and its analogs to stimulate cAMP accumulation in HKrk-B7 cells was then tested. Similar to previous experiments performed in transiently transfected COS-7 cells or stably transfected HEK293 cells (Usdin, T. B., et al., Nature Neuroscience 2:941-943 (1999); Hoare, S. R., et al., J. Biol. Chem. 275:27274-27283 (2000)), native TIP(1-39), at concentration as high a 10 μM, failed to stimulate cAMP accumulation at the PTH1R expressed in LLC-PK, cells (data not shown). A lack of second messenger formation was also observed when cells were treated with TIP(3-39), TIP(9-39), and TIP(19-39). Challenge of HKrk-B7 cells with TIP(23-39) resulted in a weak increase (˜2-fold over basal) in cAMP accumulation when added at high molar concentrations; however, a similar increase in cAMP was observed for this peptide with untransfected LLC-PK, cells (data not shown), implying that the effect was not dependent on the PTH1R.

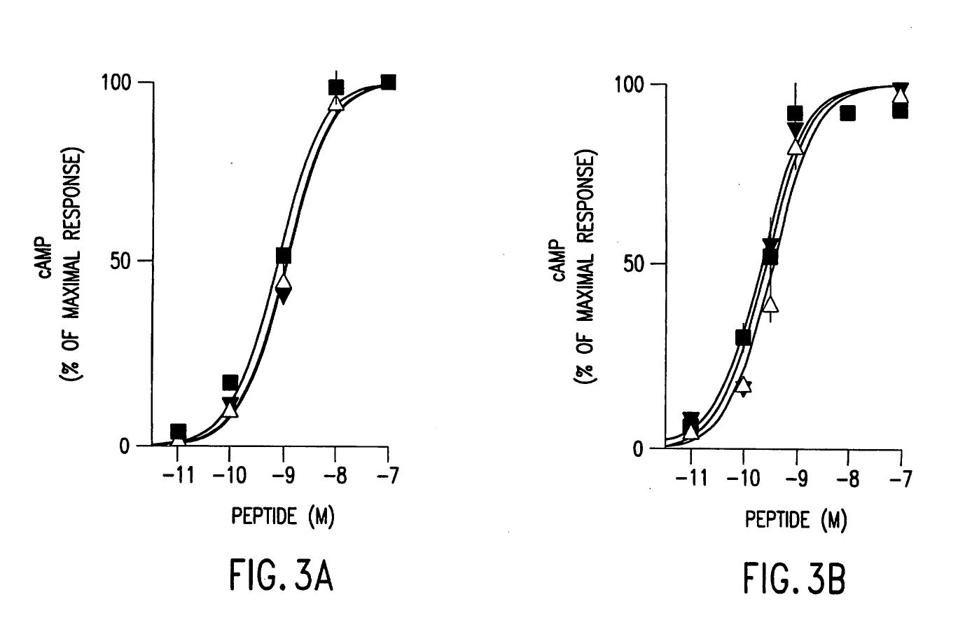

In contrast to the findings with full-length TIP(1-39) and its truncated analogs, the peptide chimera PTHrP(1-20)/TIP(23-39) was a full and potent agonist for the PTH1R and stimulated cAMP accumulation in HKrk-B7 cells with an EC₅₀ of 1.40±0.3 nM (FIG. 3A; Table 3). This potency was comparable to the EC₅₀ values observed for PTH(1-34) and PTHrP(1-36). When tested with SaOS-2 cells, an osteoblast-like cell line expressing lower levels of the PTH1R (about 30,000 receptors/cell) (Fukayama, S. and Tashjian, A. H. Jr. Endocrinology 125:1789-1794 (1989)), the PTHrP(1-20)/TIP(23-39) chimera induced cAMP accumulation with a potency (EC₅₀: 0.38±0.12 nM) which was similar to that obtained with PTH(1-34) or PTHrP(1-36) (EC₅₀ values: 0.30±0.12 and 0.25±0.15 nM, respectively) (FIG. 3B; Table 2). To begin exploring which site within the amino-terminus of TIP(1-39) prevents signal transduction at the PTH1R, three additional peptides were synthesized; the chimeras PTHrP(1-6)/TIP(9-39) and [Ile⁵]PTHrP(1-6)/TIP(9-39), as well as [Ile⁷]TIP(1-39), the latter having Asp7 replaced by the corresponding isoleucine of PTH (see FIG. 1). None of these peptides, however, stimulated cAMP formation in HKrk-B7 cells (data not shown).

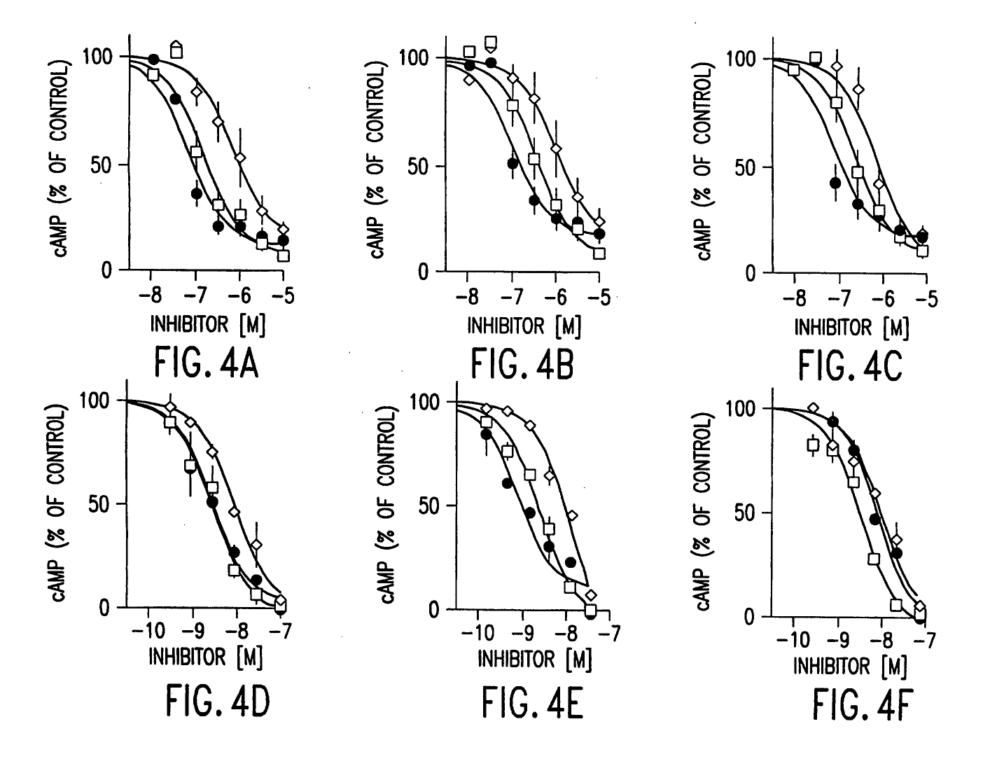

Because TIP(1-39) and some of its fragments bound to the PTH1R with high binding affinity, but lacked agonist activity, whether or not they would function as PTH1R antagonists was tested. HKrk-B7 and SaOS-2 cells were incubated with either PTH(1-34), PTHrP(1-36), or PTHrP(1-20)/TIP(23-39), at doses that would achieve an approximately half-maximal increase in cAMP accumulation, in the absence or presence of increasing concentrations of either TIP(1-39), TIP(9-39), or PTHrP(7-36) (FIG. 4A-4F). TIP(9-39) inhibited agonist-stimulated cAMP accumulation with an efficiency similar to that of PTHrP(7-36) (Carter, P. H., et al., Endocrinology 140:4972-4981 (1999)). In HKrk-B7 cells, the IC₅₀ values were=300 nM for TIP(9-39) compared to ˜100 nM for PTHrP(7-36) (FIG. 4A-4C; TIP(1-39) also functioned as an antagonist and showed a half-maximal inhibition of agonist-induced cAMP accumulation at 1000 nM. Similar results were observed in SaOS-2 cells (FIG. 4D-4F).

Table 3: Stimulation of cAMP accumulation in HKrk-B7 or SaOS-2 cells:

Cells were incubated with increasing concentrations of PTH(1-34), PTHrP(1-36), or PTHrP(1-20)/TIP(23-39) (see FIG. 3). The values for EC₅₀ and Vₘₐₓ (mean±SE) are derived from at least three independent experiments.

TABLE 3

| | HKrk-B7 | | SaOS-2 | |
|---|---|---|---|---|
| | | Max_{obs} | | Max_{obs} |
| | EC₅₀ | (pmol/ | EC₅₀ | (pmol/ |
| Ligand | (nM) | 48-well) | (nM) | 48-well) |
| [Tyr³⁴]PTH(1-34) | 0.96 ± 0.2 | 42 ± 7 | 0.30 ± 0.12 | 32 ± 5 |
| [Tyr³⁶]PTHrP(1-36) | 1.50 ± 0.2 | 38 ± 7 | 0.25 ± 0.15 | 31 ± 4 |
| PTHrP(1-20)/TIP(23-39) | 1.40 ± 0.3 | 39 ± 8 | 0.38 ± 0.12 | 31 ± 3 |

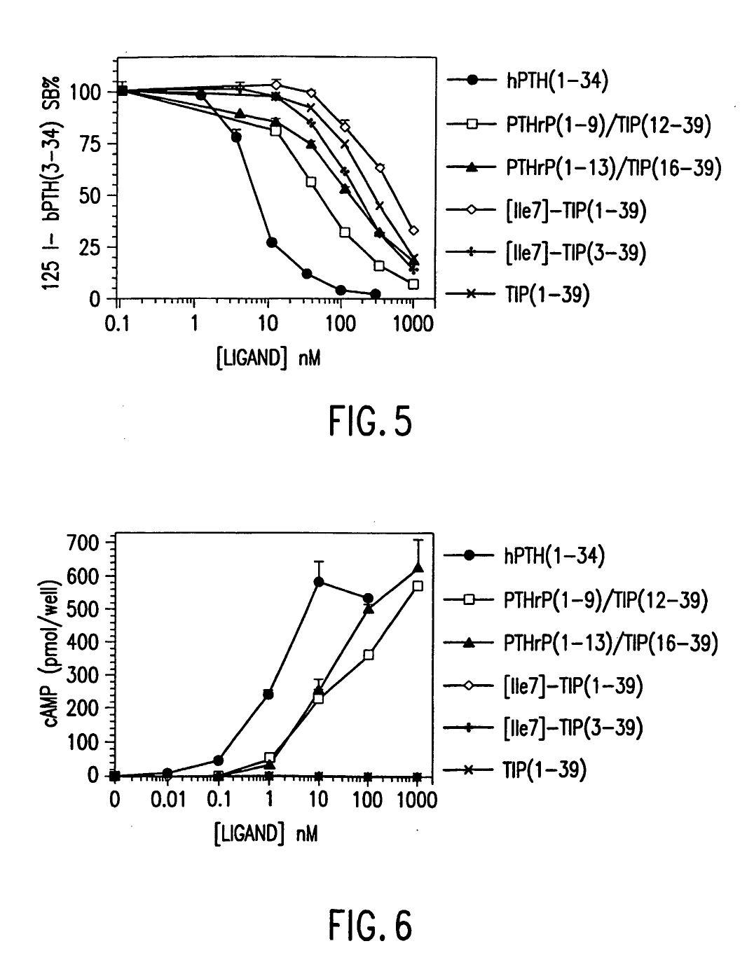

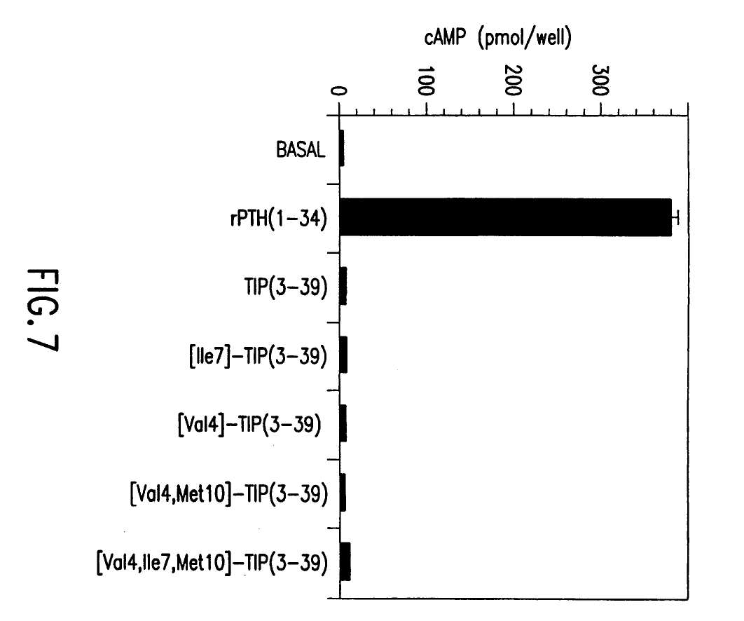

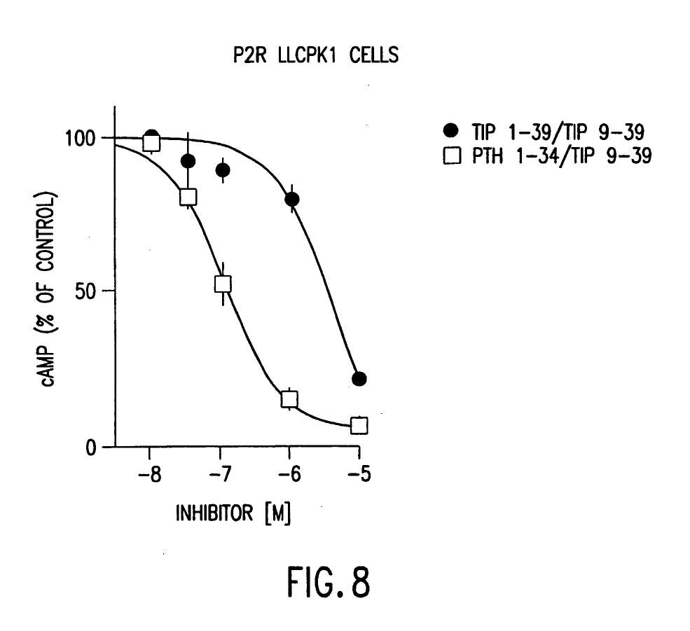

Results obtained in FIGS. 5-7 further support the above results. The results of FIG. 8 demonstrate that the truncated TIP(9-39) polypeptide is also an antagonist of the PTH2 receptor and as such should be useful in treating conditions that involve regulation of events mediated through the PTH2R.

2. Discussion

Although PTH and PTHrP share only limited amino acid sequence homology, both peptides activate the PTH1R with nearly equivalent potency and efficacy (Gardella, T. J., and Jüppner, H., “Interaction of PTH and PTHrP with their receptors,” in Reviews Endocrine Metabolic Disorders, Kluwer Academic Publisher, The Netherlands (2000), p. 317-329; Jüppner, H., et al., “Parathyroid hormone and parathyroid hormone-related peptide in the regulation of calcium homeostasis and bone development,” in DeGroot, L. J., ed., Endocrinology, W. B. Saunders, Philadelphia, Pa. (969-998 (2000)). In contrast, PTHrP is a poor stimulator of cAMP accumulation when tested with cells expressing different PTH2Rs, while PTH is able to activate at least the human PTH2R (Usdin, T. B., et al., J. Biol. Chem. 270:15455-15458 (1995); Gardella, T. J., et al., J. Biol. Chem. 271:19888-19893 (1996); Behar, V., et al., Endocrinology 137:4217-4224 (1996)). Both PTH and PTHrP, however, are poor stimulators of cAMP formation with the rat and the zebrafish PTH2R (Hoare, S. R., et al., Endocrinology 140:4419-4425 (1999); Hoare, S. R., et al., J. Biol. Chem. 275:27274-27283 (2000); Rubin, D. A., et al., J. Biol. Chem. 274:23035-23042 (1999)). Since the recently discovered hypothalamic peptide, TIP(1-39), activates all known PTH2Rs, it is likely to be the primary ligand for this receptor (Usdin, T. B., et al., Nature Neuroscience 2:941-943 (1999); Hoare, S. R. J., et al., Endocrinology 141:3080-3086 (2000)). Because of the known crossreactivity of PTH and PTHrP ligands with the PTH2R and because of the limited homology within the carboxyl-terminal regions of TIP(1-39), PTH(1-34), and PTHrP(1-36), the capacity of TIP(1-39) to interact with the PTH1R was investigated.

In contrast to PTH(1-34) and PTHrP(1-36), TIP(1-39) failed to stimulate cAMP accumulation in HKrk-B7 and SaOS-2 cells, confirming earlier studies with this peptide which had been performed in transfected COS-7 and HEK293 cells expressing the PTH1R (Usdin, T. B., et al., Nature Neuroscience 2:941-943 (1999); Hoare, S. R., et al., J. Biol. Chem. 275:27274-27283 (2000)). However, TIP(1-39) bound to the PTH1R, albeit with low affinity. To explore the structural features in TIP(1-39) that modulate the interaction with the PTH1R, several deletion mutants for receptor-binding affinity and the capacity to induce cAMP accumulation were tested. Since amino acid sequence alignment of PTH(1-34), PTHrP(1-36), and TIP(1-39) revealed that the latter peptide has an amino-terminus extended by two amino acid residues (Usdin, T. B., et al., Nature Neuroscience 2:941-943 (1999)) (see FIG. 1), it seemed plausible that this extension could account for the reduced binding affinity at the PTH1R as well as the lack of agonist activity. In fact, in comparison to TIP(1-39), TIP(3-39) exhibited a 2- to 3-fold improvement in IC₅₀ when tested with either radiolabeled rPTH(1-34) or PTHrP(1-36). However, despite the improved apparent binding affinity, this truncated analog failed to stimulate cAMP accumulation in HKrk-B7 cells; a result which is consistent with previous findings in transfected COS-7 and HEK293 cells (Usdin, T. B., et al., Nature Neuroscience 2:941-943 (1999); Hoare, S. R., et al., J. Biol. Chem. 275:27274-27283 (2000)). Thus, the first two residues of TIP(1-39) are clearly not the structural elements which prevent PTH1R activation.

Deletion of an additional six residues from the amino-terminus increased binding affinity further, as the resulting TIP(9-39) had, in comparison to TIP(1-39), a 5- to 6-fold improvement in IC₅₀. However, despite its high binding affinity, which was similar to that of the agonist PTHrP(1-36), TIP(9-39) failed to stimulate cAMP accumulation. Similarly, Hoare et al. found that TIP(7-39) efficiently inhibited radioligand binding to the PTH1R, but showed no agonist activity (Hoare, S. R., et al., J. Biol. Chem. 275:27274-27283 (2000), Hoare and Usdin, J. Pharmacology Exp. Ther. 295:761-770 (2000)). Therefore, TIP(1-39) and TIP(9-39) were directly tested for their antagonist activity on the PTH1R.

Analogs of PTH and PTHrP that are the most potent in vivo antagonists comprise the amino acid sequence 7-34 or 7-36, with or without activity enhancing amino acid modifications. Since TIP39, when aligned with PTH and PTHrP (see FIG. 1) appears to have an amino-terminal extension of two amino acid residues, a TIP39 analog was synthesized that had a truncation of the first eight residues, i.e. at those residues in PTH and PTHrP that yielded potent in vitro and in vivo antagonists.

TIP(9-39) was able to inhibit the actions of PTH(1-34), PTHrP(1-36), or PTHrP(1-20)/TIP(23-39) with a potency similar to that of PTHrP(7-36) (Carter, P. H., et al., Endocrinology 140:4972-4981 (1999)). Taken together, our findings suggest that the carboxyl-terminal regions of three different peptides share sufficient structural homology to allow efficient binding to the same or similar sites in the PTH1R. Consistent with this conclusion, a recent NMR study of TIP(1-39) revealed a secondary structure profile that was similar to that of PTH(1-34), i.e. two α-helices connected by flexible linker regions of yet undefined structure (Piserchio, A., et al., J. Biol. Chem. 275:27284-27290 (2000)).

Previous studies by the inventors and others have led to the conclusion that the interaction of PTH(1-34) and PTHrP(1-36) with the PTH1R involves two distinct principal receptor components (for review see Gardella, T. J., and Jüppner, H., “Interaction of PTH and PTHrP with their receptors,” in Reviews Endocrine Metabolic Disorders, Kluwer Academic Publisher, The Netherlands (2000), p. 317-329). According to this model, which is supported by several different cross-linking studies (Zhou, A. T., et al., Proc. Natl. Acad. Sci. USA 94:3644-3649 (1997); Mannstadt, M., et al, J. Biol. Chem. 273:16890-16896 (1998); Bisello, A., et al., J. Biol. Chem. 273:22498-22505 (1998); Behar, V., et al., J Biol. Chem. 275:9-17 (2000); Carter, P. H., et al., “Full-length photolabile analogs of PTH-related peptide are inverse agonists with and crosslink to constitutively active PTH-1 receptors,” The Endocrine Society's 81st Annual Meeting, San Diego, Calif. (1999), p. P2-627), the carboxyl-terminal regions of PTH(1-34) and PTHrP(1-36) interact predominantly with the amino-terminal, extracellular domain of the PTH1R to provide binding energy, while the amino-terminal portion of either ligand then interacts with the receptor's membrane-spanning helices and the connecting extracellular loops to induce signal transduction. Fragments of TIP(1-39) that are truncated at the amino-terminus, i.e. TIP(3-39) and TIP(9-39), bound to the PTH1R with reasonably high affinity, and at least TIP(9-39) inhibited the actions of PTH(1-34), PTHrP(1-36), and the PTHrP/TIP chimera, as efficiently as PTHrP(7-36) (Carter, P. H., et al., Endocrinology 140:4972-4981 (1999)). Taken together with the observation that PTHrP(1-20)/TIP(23-39) activated the PTH1R as efficiently as PTH(1-34) and PTHrP(1-36), it appears likely that the interaction between the PTH1R and TIP(1-39) involves residues in the ligand's carboxyl-terminus and the receptor's amino-terminal, extracellular domain. This hypothesis is supported by recent observations by Hoare et al., who demonstrated that a PTH1R/PTH2R chimera (containing the amino-terminal, extracellular domain and the first membrane-spanning helix of the PTH1R fused to the remaining portions of the PTH2R), but not the reciprocal PTH2R/PTH1R chimera, is efficiently activated by TIP(1-39) (Hoare, S. R., et al., J. Biol. Chem. 275:27274-27283 (2000)).

In our studies, TIP(19-39) and TIP(23-39) showed no detectable binding to the PTH1R, even though this portion of TIP contains several amino acid residues that are functionally important in PTH(11-34) or PTHrP(1-36), i.e. Glu21, Arg22, Arg23, Trp25, and Leu26 (Gardella, T. J., and Jüppner, H., “Interaction of PTH and PTHrP with their receptors,” in Reviews Endocrine Metabolic Disorders, Kluwer Academic Publisher, The Netherlands (2000), p. 317-329; Mannstadt, M., et al., J. Biol. Chem. 273:16890-16896 (1998); Gardella, T. J., et al., Endocrinology 135:1186-1194 (1994)). (see FIG. 1). Previous investigations had indicated that PTH(15-34)amide binds with very low (micromolar) affinity to the PTH1R (Jüppner, H., et al., Endocrinology 134:879-884 (1994)), and it is therefore not too surprising that TIP analogs comprising only the most carboxyl-terminal portion of the ligand exhibited no detectable binding to this receptor. These results furthermore imply that the region 9-18 of TIP(1-39) contributes to binding affinity. Because TIP(1-39) showed antagonist activity at the PTH1R, it conceivably could act as an endogenous inhibitor of PTH and/or PTHrP action at the PTH1R, if it were to be secreted into the circulation at sufficiently high concentrations. Conversely, synthetic PTH and PTHrP analogs that bind to the PTH1R could have unwarranted effects in those tissues where the PTH2R is most abundantly found (Usdin, T. B., et al., J. Biol. Chem. 270:15455-15458 (1995); Usdin, T. B., et al., Endocrinology 137:4285-4297 (1996)).

The amino-terminal domain of TIP(1-39) is likely to be positioned at least near the activation pocket of the PTH1R when bound to this receptor, but remains uncertain what prevents its activation. The lack of activation is clearly not related to presence of the two amino acid extension at the amino-terminus (this study and (Hoare, S. R., et al., J. Biol. Chem. 275:27274-27283 (2000))), however several other candidate residues in the amino-terminal region of TIP(1-39) might be involved. Most substitutions in the 1-9 region of PTH have been recently shown to impair PTH1R activation (Shimizu, M., et al., J. Biol. Chem. 275:21836-21843 (2000)), and it may well be that one or more of the divergent residues in the corresponding region of TIP(1-39), i.e. Asp7, Ala8, Ala9, Phe10, and Arg11, prevent a productive interaction with the PTH1R. It is furthermore likely that one or several of the first eight ligand residues impair PTH1R-binding affinity (see Tabl. 1 and Hoare, S. R., et al., J. Biol. Chem. 275:27274-27283 (2000)). While the underlying mechanisms are unknown, a recent computer modeling study of the ligand-receptor complex has suggested that some of the amino-terminal residues of TIP(1-39), such as Asp7, would not fit productively into the agonist-binding pocket of the PTH1R (Piserchio, A., et al., J. Biol. Chem. 275:27284-27290 (2000)). Because Asp7 aligned with PTH residue Ile5, which determines PTH2R agonist selectivity (Jüppner, H., et al., “Parathyroid hormone and parathyroid hormone-related peptide in the regulation of calcium homeostasis and bone development,” in DeGroot, L. J., ed., Endocrinology, W. B. Saunders, Philadelphia, Pa. (996-998 (2000); Gardella, T. J., et al., J. Biol. Chem. 271:19888-19893 (1996); Behar, V., et al., Endocrinology 137:4217-4224 (1996)), this residue was replaced with Ile. However, no activation of the PTH1R was observed with [Ile⁷]TIP(1-39) (data not shown). Taken together with the lack of PTH1R activation by PTHrP(1-6)/TIP(9-39) and by [Ile⁵]PTHrP(1-6)/TIP(9-39), it thus appears likely that other divergent amino acid residues in the amino-terminal region of TIP(1-39) are involved in preventing agonist actions at the PTH1R. It should be possible to identify these residues through the development of additional TIP chimeras and analogs, and to further define the structural basis of the TIP/PTH1R interaction. The resulting information is likely to provide additional new insights into functionally important regions of the PTH1R, and these may help in the development of receptor-specific agonists and/or antagonists.

### SUMMARY

The tuberoinfindibular peptide TIP39 (TIP(1-39)), which exhibits only limited amino acid sequence homology with PTH and PTHrP, stimulates cAMP accumulation in cells expressing the PTH2-receptor (PTH2R), but it is essentially inactive at the PTH/PTHrP receptor (PTH1R). However, when using either ¹²⁵I-labeled rat [Nle^{8,21}, Tyr³⁴]PTH(1-34)amide (rPTH) or ¹²⁵I-labeled human [Tyr³⁶]PTHrP(1-36)amide (PTHrP(1-36)) for radioreceptor studies, TIP(1-39) bound to LLCPK₁ cells stably expressing the PTH1R (HKrk-B7 cells), albeit with weak apparent affinity (243±52 nM and 210±64 nM, respectively). In comparison to the parent peptide, the apparent binding affinity of TIP(3-39) was about 3-fold higher and that of TIP(9-39) was about 5.5-fold higher. However, despite their improved IC₅₀ values at the PTH1R, both truncated peptides failed to stimulate cAMP accumulation in HKrk-B7 cells. In contrast, the chimeric peptide PTHrP(1-20)/TIP(23-39) bound to HKrk-B7 cells with affinities of 31±8.2 nM and 11±4.0 nM when using radiolabeled rPTH and PTHrP(1-36), respectively, and it stimulated cAMP accumulation in HKrk-B7 and SaOS-2 cells with potencies (EC₅₀: 1.40±0.3 nM and 0.38±0.12 nM, respectively) and efficacies (Vₘₐₓ: 39±8 pmol/well and 31±3 pmol/well, respectively) that were similar to those of PTH(1-34) and PTHrP(1-36). In both cell lines, TIP(9-39), and to a lesser extent TIP(1-39), inhibited the actions of the three agonists with efficiencies that were similar to those of [Leu¹¹, D-Trp¹², Trp²³, Tyr³⁶]PTHrP(7-36)amide, an established PTH1R antagonist. Taken together, the currently available data suggest that the carboxyl-terminal portion of TIP(1-39) interacts efficiently with the PTH1R, at sites that are identical or closely overlapping with those utilized by PTH(1-34) and PTHrP(1-36). The amino-terminal residues of TIP(1-39), however, are unable to interact productively with the PTH1R, thus enabling TIP(1-39) and some of its truncated analogs to function as an antagonist at this receptor.

### EXAMPLE 2

### Therapeutic Use of Truncated TIP9-39

The above studies indicate that TIP(9-39) may be as potent as PTH(7-34) or PTHrP(7-34 or 6) (with or without activity enhancing amino acid modifications) when administered in vitro and therefore possibly in vivo. It thus appears likely that TIP(9-39), analogs thereof, or peptide that are further truncated at the amino-terminus, could gain importance in the treatment of hypercalcemia caused by hyperparathyroid conditions and/or humoral hypercalcemia of malignancy. Furthermore, since PTHrP(1-20)/TIP(23-39) shows an efficacy in vitro that is equivalent to that of PTH(1-34) and PTHrP(1-36) it appears likely that this chimeric peptide will show an equivalent potency when tested in vivo. PTHrP(1-20)/TIP(23-39), similar chimeras between PTH and TIP39, or chimeras with different lengths of either peptide component, are thus likely to display a similar efficacy for the treatment of osteoporosis or related disorders as analogs of PTH and PTHrP.

The invention provides a method for treating a medical disorder that results from altered or excessive action of the PTH/PTHrP receptor, comprising administering to a patient a therapeutically efficient amount of a TIP39 polypeptide, such as for example TIP9-39, sufficient to inhibit activation of the PTH/PTHrP receptor of said patient.

In this embodiment, a patient who is suspected of having a disorder resulting from altered action of the PTH/PTHrP receptor may be treated using the those peptide analogs of the invention shown to be selective antagonists of the PTH/PTHrP receptor. Such antagonists include the compounds of the invention which have been determined (by the assays described herein) to interfere with PTH/PTHrP receptor-mediated cell activation or other analogs having similar properties.

To administer the antagonist, the appropriate peptide is used in the manufacture of a medicament, generally by being formulated in an appropriate carrier or excipient such as, e.g., physiological saline, and administered intravenously, intramuscularly, subcutaneously, or orally, at a dosage that provides adequate inhibition of PTH binding to the PTH/PTHrP receptor. Typical dosage would be 1 ng to 10 mg of the peptide per kg body weight per day.

The invention also provides a method for treating conditions characterized by bone loss, such as for example osteoporosis. A patient is treated with a therapeutically efficient amount of a chimeric polypeptide such as the PTH1R agonist comprising the sequence of the chimeric polypeptide PTHrP(1-20)/TIP(23-39) (AVSEHQLLHDKGKSIQDLRRRHWLNSYMHKLLVLDAP) [SEQ ID NO:8].

### EXAMPLE 3

### Identification and Characterization of the Murine and Human Gene Encoding the Tuberoinfundibular Peptide of 39 Residues (TIP39)

The tuberoinfundibular peptide of 39 residues (TIP39) was recently purified from bovine hypothalamic extracts and the complete amino acid sequence of the mature peptide was obtained by microsequence analysis (Usdin, T. B., et al., Nat. Neurosci. 2:941-943 (1999)). TIP39 appears to be distantly related to parathyroid hormone (PTH) and PTH-related peptide (PTHrP), since nine amino acid residues, some of which have been shown to be functionally important in both latter peptides (Gardella, T. J., et al., J. Biol. Chem. 271:19888-19893 (1996)), are either conserved or identical among all three peptides. Although PTH was shown to efficiently activate the human type 2 PTH receptor (PTH2 receptor) (Gardella, T. J., et al., J. Biol. Chem. 271:19888-19893 (1996); Usdin, T. B., et al., J. Biol. Chem. 270:15455-15458 (1995); Behar, V., et al., Endocrinology 137:4217-4224 (1996)), this peptide was later shown to interact only weakly with rat and zebrafish homologs of this receptor (Hoare, S. R., et al., Endocrinology 140:4419-4425 (1999); Rubin, D. A., et al., J. Biol. Chem. 274:23035-23042 (1999)). Furthermore, PTHrP activated none of the known PTH2 receptor homologs, although this peptide was shown to bind, albeit with reduced affinity, to this subfamily of receptors (Gardella, T. J., et al., J. Biol. Chem. 271:19888-19893 (1996); Behar, V., et al., Endocrinology 137:42174224 (1996); Clark, J. A., et al., Mol. Endocrinol. 12:193-206 (1998)). Because this finding suggested that PTH and PTHrP are not the primary ligand for the PTH2 receptor, a search for a novel agonist at this receptor was began. Initial studies revealed that bovine hypothalamic extracts, which failed to activate the PTH/PTHrP receptor, efficiently stimulated cAMP accumulation in cells expressing the rat or the human PTH2 receptor (Usdin, T. B., Endocrinology 138:831-834(1997)). Subsequent efforts led to the isolation and definition of the primary structure of a novel peptide, referred to as TIP39, from bovine hypothalamus and the synthetic peptide was shown to efficiently activate human, rat, and zebrafish PTH2 receptors, but not PTH/PTHrP receptors from several different species (Usdin, T. B., et al., Nat. Neurosci. 2:941-943 (1999); Hoare, S. R. J., et al., Endocrinology 141:3080-3086 (2000); Jonsson, K. B., et al, Endocrinology 142:704-709 (2001)). TIP39 rather than PTH (or PTHrP) thus appeared to be the primary ligand for the PTH2 receptor. However, native TIP39 and some of its amino-terminally truncated analogs were shown to bind to the PTH/PTHrP receptor and to act as competitive antagonists of PTH- or PTHrP-stimulated cAMP accumulation (Jonsson, K. B., et al., Endocrinology 142:704-709(2001); Hoare, S. R., et al, J. Biol. Chem. 275:27274-27283 (2000)). Three distinct peptides, PTH, PTHrP and TIP39 that share only limited amino acid sequence homology thus interact with the PTH/PTHrP receptor.

Little is thus far known about the physiologic role(s) of the TIP39-PTH2 receptor system. The PTH2 receptor is expressed in somatostatin-expressing hypothalamic periventricular neurons, which suggested a possible role in the regulation of growth hormone release (Usdin, T. B., et al., Nat. Neurosci. 2:941-943 (1999)). It is also expressed in the spinal cord, within the superficial layers of the dorsal horn, indicating that TIP39 may be involved in pain perception (Usdin, T. B., et al., Nat. Neurosci 2:941-943 (1999)). Furthermore, TIP39 may be identical or related to a hypothalamic substance that stimulates renin release in the juxta-glomerular apparatus of the kidney (Urban, J., et al., Neuroendocrinology 55:574-582 (1992)), where the PTH2 receptor is expressed (Usdin, T. B., et al., Endocrinology 137:4285-4297 (1996)), and may thus have a role in blood pressure regulation.

Reported herein is the identification of genomic DNA sequences encoding human and murine TIP39, the organization of both mammalian genes, and a partial functional characterization of the mature peptides from both species. Furthermore, an initial assessment of the tissue distribution of mouse TIP39 mRNA, and of the phylogenetic relationship between TIP39, PTH and PTHrP is provided.

Materials and Methods

Identification of Genomic Clones Encoding Human and Mouse TIP39, Chromosomal Location of Their Genes, and Predictions Regarding the Cleavage of the Precursor Peptides

Partial genomic nucleotide sequence encoding TIP39 was obtained by searching the high throughput genomic sequence (htgs) draft sequences of the National Center for Biotechnology Information (NCBI) with the bovine TIP39 amino acid sequence (TBLASTN search). Nucleotide sequence alignment of human and murine genomic DNA encoding TIP39 was performed using the NCBI Blast 2 sequences server with default parameters (http://www.ncbi.nlm.nih.gov/gorf/bl2.html), the GCG Wisconsin Package, or MacVector 7.0 software (both from Genetics Computer Group, Madison, Wis., USA). Information regarding the chromosomal localization of human TIP39 was obtained by searching the database of the Genome Sequencing Center at Washington University School of Medicine, St. Louis, Mo., USA with nucleotide sequence information from different BAC clones (http://genome.wustl.edu/gsc/human/Mapping/index.shtml). Additional sequence information was obtained by using the Human Genome Browser of the University of Santa Cruz, Calif., USA (http://genome.cse.ucsc.edu) and the Ensembl Genome Server of the EMBL European Bioinformatics Institute (http://www.ensembl.org). Putative cleavage sites within the TIP39 precursor were predicted using the neural network approach of SignalP V2.0b2 of the Center for Biological Sequence Analysis, BioCentrum-DTU, Technical University of Denmark (http://www.cbs.dtu.dk/services/SignaIP-2.0/) (Nielsen, H., et al., Protein Engineering 10:1-6 (1997); Nielsen, H., et al., Protein Engineering 12:3-9 (1999)). This program was also used to predict cleavage sites for human PTH and PTHrP, which allowed verification of the computer program through previously published experimental data (Yasuda, T., et al., J. Biol. Chem. 264:7720-7725 (1989); Wiren, K. M., et al., J. Biol. Chem. 263:19771-19777 (1988)).

Peptides

Human TIP-(1-39) and TIP-(9-39), mouse TIP-(1-39), [Tyr³⁴] human PTH-(1-34)amide (PTH-(1-34)) were synthesized by the Biopolymer Core Facility at Massachusetts General Hospital (Boston, Mass.) using Fmoc chemistry on Perkin-Elmer Applied Biosystems synthesizers (model 430A or 431A). All peptides were purified to homogeneity by reversed-phase chromatography, and amino acid sequences were confirmed by analysis of amino acid composition and amino acid sequence, and by mass spectroscopy.

Cell Culture and Stimulation of cAMP Accumulation

DMEM, Trypsin/EDTA, penicillin G/streptomycin, and horse serum were from Gibco/BRL, Life Technologies, Gaithersburg, Md. LLCPK₁ cells expressing the human PTH2 receptor, clone hPR2-20 (approximately 0.8×10⁶ copies/cell), were kindly provided by F. R. Bringhurst, Endocrine Unit, Massachusetts General Hospital, Boston, Mass. Cells were maintained in DMEM supplemented with 10% heat-inactivated fetal bovine serum, 100 U/ml penicillin, and 100 μg/ml streptomycin, in a humidified atmosphere containing 95% air and 5% CO₂, as previously described (Gardella, T. J., et al., J. Biol. Chem. 271:19888-19893 (1996); Carter, P. H., et al., Endocrinology 140:4972-4981 (1999)). After seeding into 48-well plates, medium was replaced every other day. Upon confluency, cells were used for stimulating cAMP accumulation. Agonist-dependent stimulation of cAMP accumulation was performed at room temperature for 45 minutes, and the subsequent measurement of cAMP by radioimmunoassay was performed as previously described (Gardella, T. J., et al., J. Biol. Chem. 271:19888-19893 (1996); Bergwitz, C., et al., Endocrinology 139:723-732 (1998)). Data were analyzed and graphically displayed using the Prism 3.0 software package (GraphPad Software, Inc., San Diego, Calif.).

Rapid Amplification of cDNA Ends (RACE)

To identify the 5′ end of the cDNA encoding human TIP39, 5′ RACE was performed by using a Marathon-Ready cDNA kit to amplify cDNAs from human hypothalamus (CLONTECH). The initial PCR was performed using the provided API primer and a primer specific for human TIP39 (hTIPr5: 5′-AGCAGCTTGTGCATGTACGAG-3′)[SEQ ID NO:15]. A 50 μl reaction consisted of 5 μl hypothalamic cDNA, 1 μl API primer, 1 μl hTIPr5 primer (100 pmol), 1 μl (2U) polymerase (GC-rich polymerase, ROCHE), 1 μl dNTPs (10 mM each, ROCHE), 10 μl PCR-Buffer, 5 μl GC-rich solution, and 31 μl H₂O. The following optimized reaction profile was carried out using an Eppendorf Mastercycler: initial denaturation at 98° C. for 1 minute and at 95° C. for 2 minutes; subsequent program: denaturation at 95° C. for 30 seconds, annealing at 69° C. for 30 seconds, polymerization at 72° C. for 2 minutes; after the first cycle, the annealing temperature was decreased by 1° C. for each of the following 4 cycles. Subsequently, 35 cycles were performed with denaturation at 95° C. for 2 minutes, annealing at 63° C. for 30 seconds, and polymerization at 72° C. for 2 minutes; final extension at 72° C. for 7 minutes. 5 μl of the diluted product (1:50 in H₂O) was reamplified in a nested PCR using 1 μl AP2 primer, 1 μl hTIPr4 primer (5′-TTGTGCATGTACGAGTTCAGC-3′ [SEQ ID NO:16]; 100 pmol), and the same reaction profile as before. This reaction was electrophoresed through a 2% agarose gel and stained with ethidium bromide. For molecular cloning, 4 μl of the final PCR product were ligated into pCR 2.1-TOPO (Invitrogen) for transformation of TOP10 cells. Plasmid DNA was prepared by standard techniques and DNA sequence analysis was performed at the Massachusetts General Hospital core sequencing facility.

RT-PCR

Approximately lug of poly-A⁺ RNA from murine brain (Ambion) was reverse transcribed using a primer specific for murine TIP39 (mTIP2rev: 5′-GTCCAGTAGCAACAGCTTCTGC-3′ [SEQ ID NO:17]; 100 pmol) and the Omniscript II reverse transcriptase kit (Qiagen) at 42° C. for 1 hour (final reaction volume: 20%1). One tenth of the reaction was used for an initial PCR, which consisted of 2 μl (100 ng) reverse transcribed template DNA, 1 μl dNTPs (10 mM each, ROCHE), 1 μl (100 pmol) mTIPCR2-f6 forward primer (5′-TCTCTATTTTTATCCCTCTGAC-3′; 100 pmol)[SEQ ID NO:18], 1 μl (100 pmol) mTIP2rev primer, 5 μl PCR-Buffer (Qiagen), 10 μl Q-solution (Qiagen), 0.5 μl HotStar Taq polymerase (Qiagen) and 29 μl H₂O. The reaction profile was: initial denaturation at 95° C. for 15 minutes, then 35 cycles with denaturation at 94.5° C. for 30 seconds, annealing at 65° for 45 seconds, polymerization at 72° C. for 30 seconds; final extension at 72° C. for 10 minutes. A nested PCR using 2 μl of the initial reaction product was performed using forward primer mTIPCR2-f5 (5′-CTCTGACACACCCCTTGTGTC-3′ [SEQ ID NO:19]; 100 pmol) and reverse primer mTIP2rev following the same reaction profile. 4 μl of the final reaction product were ligated into pCR 2.1-TOPO (Invitrogen) for transformation of TOP10 cells.

Preparation of a cDNA Encoding Portions of Murine TIP39

A 103 bp genomic DNA fragment encoding portions of murine TIP39 was PCR-amplified using the following reaction profile: 1 μl (200 ng) mouse genomic DNA, 1 μl mTIP5 for primer (5′-CTAGCTGACGACGCGGCCTTTCG-3′ [SEQ ID NO:20]; 100 pmol), 111 mTIP2rev primer (100 pmol), 1 μl dNTPs (10 mM, ROCHE), 5 μl PCR buffer, 1 μl DMSO, 0.5 μl Pfu-Turbo polymerase (Stratagene), and 39.5 μl H₂O; initial denaturation at 98° C. for 1 minute and at 95° C. for 3 minutes, denaturation at 95° C. for 45 seconds, annealing at 69.5° for 1 minute, polymerization at 72° C. for 30 seconds; after the first cycle, the annealing temperature was decreased by 1° C. for each of the following 4 cycles; subsequently, 35 cycles with denaturation at 95° C. for 45 seconds, annealing at 64.5° C. for 1 minute, polymerization at 72° C. for 30 seconds; final extension at 72° C. for 5 minutes. The reaction was electrophoresed through a 4% agarose gel and stained with ethidium bromide. 40 μl of the reaction were purified using the QIAquick PCR purification kit (Qiagen) and eluted with 30 μl H₂O. 4 μl of the eluate was ligated into pCR 4Blunt-TOPO (Invitrogen) for transformation of TOP10 cells. Nucleotide sequence and orientation of the insert was confirmed by nucleotide sequence analysis using a M13 reverse primer (Massachusetts General Hospital, core sequencing facility).

Northern Blot Analysis

A mouse multiple tissue Northern blot (Clontech, Palo Alto, Calif.) with 2 μg poly(A)⁺-RNA from eight different tissues was probed with the cDNA encoding mouse TIP39 (nucleotides 1 to 472; AY048587). After excision from the vector using EcoR1 (New England Biolabs, Beverly, Mass.) and purification, approximately 50 ng of the cDNA encoding TIP39 were random-labeled with ³²P-dCTP using the Prime-a-Gene labeling system (Promega, Madison, Wis.). The blot was prehybridized in 5 ml of ExpressHyb hybridization solution (Clontech, Palo Alto, Calif.) (72° C. for 3 hours) and hybridized with 5 ml of hybridization solution containing the labeled probe (1.5 hours at 72° C.). Four 15 minutes washes with 2×SSC, 0.1% SDS were performed at room temperature; subsequently two washes, 20 minute each, were performed with 0.1×SSC, 0.1% SDS at 50° C. and the blot was exposed for 3 days using Kodak X-OMAT AR flims (Kodak, Rochester, N.Y.).

In Situ Hybridization

Fresh frozen tissue sections were prepared from 10-12 week-old adult mice; 10 μm tissue sections were mounted on superfrost plus microscope slides (Fisher Scientific, Pittsburgh, Pa.) and stored at −80° C. until hybridization. The hybridization procedure was performed as described (Arai, M., and Kwiatkowski, D. J., Dev. Dyn. 215:297-307 (1999)) using complementary ³⁵S-labeled riboprobes (complementary RNAs, cRNAs). The antisense probe was transcribed from the pCR 4Blunt plasmid comprising 103 bp of murine TIP39 (see above) using the T3 polymerase; the sense probe, which served as negative control, was transcribed from the same plasmid using the T7 polymerase. Slides were covered with Kodak NTB-2 emulsion (Rochester, N.Y.) and exposed for 2-4 weeks, before developing and staining with hematoxylin and eosin (Arai, M., and Kwiatkowski, D. J., Dev. Dyn. 215:297-307 (1999)). Electronic images were obtained with both bright and dark field optics using a Nikon photomicroscope.

Phylogenetic Analysis

To further explore whether TIP39 is related to PTH and PTHrP, phylogenetic analyses were performed using all currently available species of these three peptides. With the exception of equine PTH and bovine TIP39 for which precursor sequences were not available, complete amino acid sequences that included the signal peptides were used for alignment by CLUSTAL W (Higgins, D. G., et al., Methods Enzymol. 266:383402 (1996)). These aligned amino acid sequences were subsequently entered into MacClade 4.0 (Maddison, W. P., and Maddison, M. D., MacClade 4.0: Analysis of phylogeny and character evolution (Fourth Edition), Sinauer Associates, Sunderland, Mass. (2000)) with manual adjustments as described (Dores, R. M., et al., Gen. Comp. Endocrinol. 103:1-12 (1996)). These data were analyzed for either distance (Neighbor-Joining) or parsimony (Maximum Parsimony) using PAUP version 4.0b8 (Swofford, D. L., PAUP*. Phylogenetic analysis using parsimony (* and other methods), Sinauer Associates, Sunderland, Mass. (2000)). For each analysis, 10,000 bootstrap and jackknife replicates were carried out on the entire set, in which the human gastrointestinal-inhibitory peptide (GIP) was used as the outgroup, while secretin (human, pig, and mouse), vasoactive intestinal peptide (VIP, human, mouse, and chicken), and all known homologs of PTH, PTHrP, and TIP39 formed the ingroups. Analysis was performed also on a modified data set lacking the signal peptides.

Results and Discussion

Identification of BAC Clones Encoding Human and Mouse TIP39

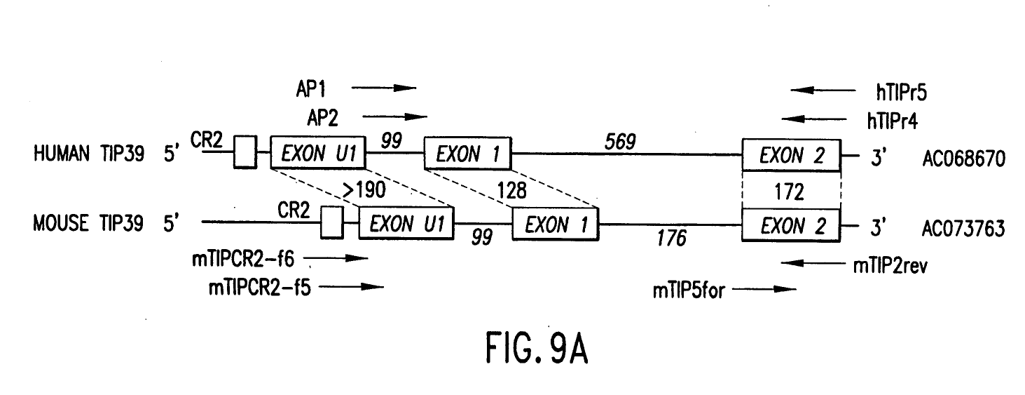

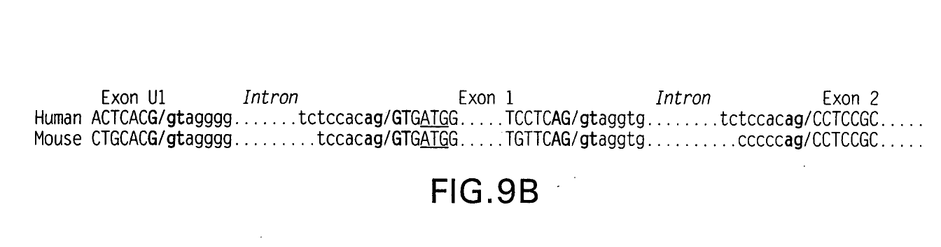

TBLASTN homology searches of the GenBank Nucleotide Sequence database (HTGS, draft sequences) were performed using the entire amino acid sequence of bovine TIP39. We identified two unordered BAC clones, one human clone (accession no: AC068670) encoding a peptide that was 100% identical to secreted bovine TIP39, and one mouse clone (accession no: AC073763) encoding a peptide that showed four amino acid differences when compared to the human/bovine TIP39 amino acid sequence (FIG. 9A). No additional genomic sequences encoding peptides with significant amino acid sequence homology to human/bovine TIP39 were identified.

Searching the database of the Genome Sequencing Center at Washington University School of Medicine, St. Louis, Mo., USA (http://genome.wustl.edu/gsc/human/Mapping/index.shtml) with clone AC068670 revealed that the genetic locus for human TIP39 resides on chromosome 19q13.33. This clone is flanked towards the centromer by the fully sequenced and assembled BAC clone AC024079.2, and towards the telomer by the fully sequenced and assembled clone AC011495.6; it partially overlaps furthermore with the finished clones AC011450.4, AC008891.7, AC010524.6 and AC010643.5, and with the unordered clones AC068786.11, and AC010619.5. Within this genetic region are several microsatellite markers, including D19S987 and D19S669E, which are located centromeric and telomeric of TIP39, respectively. A total of 70 single nucleotide polymorphisms (SNPS) in BAC clone AC068670 are currently available from dbSNP, which may also be helpful for genetic linkage studies. The mouse genomic region corresponding to human chromosome 19q13.33 is located on mouse chromosome 7.

Gene Structure and cDNA Encoding TIP39

To determine the intron-exon structure of the murine and human TIP39 gene, we first aligned genomic DNA fragments derived from human BAC clone AC068670 and mouse BAC clone AC073763. The alignment of two 2150 bp fragments from these clones revealed four regions of particularly high nucleotide identity between mouse and human genomic DNA, while the intervening sequences showed less nucleotide sequence identity (FIG. 9A). The first region, referred to as CR2, contained 55 bp and showed 96% nucleotide sequence identity between mouse and human genomic DNA. A second conserved region located 34 bp down-stream of CR2, contained 145 bp which showed 81% nucleotide sequence identity and was subsequently found to comprise portions of exon U1. Two additional regions comprising 207 bp and 180 bp showed 82% and 81% nucleotide sequence identity; these regions were subsequently found to contain exons 1 and 2 of the mouse and the human TIP39.

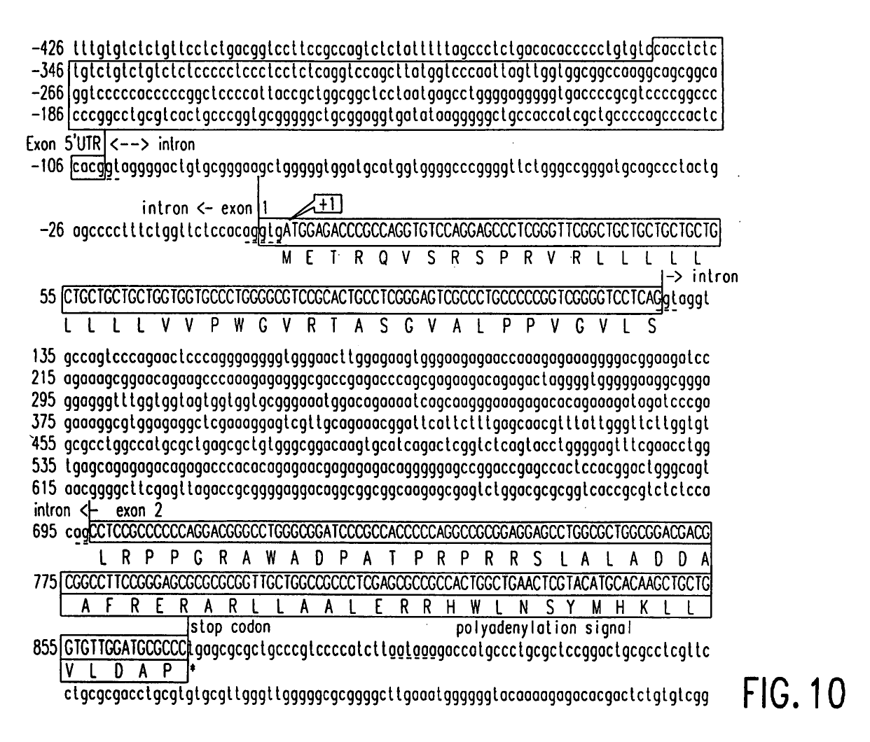

For both mammalian species, the most 3′ region (exon 2) contained an open reading frame (ORF) encoding the entire secreted TIP39, followed by a consensus sequence for polyadenylation that is located in both mammalian genes 21 nucleotides down-stream of the termination codon. Fifty-four nucleotides further up-stream of the sequences encoding the mature TIP39s, potential splice sites were identified in both species and the nucleotide sequence identity decreased thereafter. The next region with higher nucleotide sequence homology (exon 1) contained in both species an ORF encoding a putative initiator methionine (residue −61) and a stretch of thirty hydrophobic amino acids (residues −51 to −22) that could serve as leader sequences; for mouse and human genomic DNA these ORFs were flanked by nucleotide sequences possibly representing splice sites (FIG. 9B [SEQ ID NOS: 22-29] and FIG. 10 [SEQ ID NO:30]). Based on these findings, the mouse and human cDNAs were both predicted to encode TIP39 precursors comprising 100 amino acids. However, it remains uncertain whether additional exons exist which could give rise to alternatively spliced mRNAs that are either larger or smaller in size, and encode peptides that differ in size (see below).

To confirm these predictions regarding the size of the mammalian cDNAs, to identify possibly untranslated exons, and to investigate whether several differently spliced mRNAs are derived from the two mammalian TIP39 genes, 5′ RACE using commercially available adult human hypothalamic cDNA and RT-PCR using murine brain poly-A⁺ RNA was performed. Only a single product of approximately 310 bp encoding human TIP39 was obtained by nested PCR amplification of human hypothalamic cDNA (using primers “AP2” and “hTIPr4”, see FIG. 9A). Nucleotide sequence analysis of this PCR product confirmed that human TIP39 is indeed encoded by two exons (exons 1 and 2), i.e. the 569 bp of intronic nucleotide sequence predicted based on the comparison between murine and human TIP39 had been excised. However, since the PCR product derived from the human hypothalamic cDNA library contained only seven novel basepairs at the 5′ end (which furthermore represented consensus splice sequences), no information regarding non-coding sequences upstream of the putative initiator AUG was available. Additional PCRs using the human hypothalamic cDNA and primers located further up-stream failed to provide additional 5′ untranslated nucleotide sequence.

Total mRNA was reverse transcribed from mouse brain with primer mTIP2rev and nested PCRs were performed using this primer and additional mouse-specific forward primers located in those genomic regions that showed the highest nucleotide sequence homology when comparing mouse and human genomic DNA (CR2 and exon U1) (see FIG. 9A). When using primers mTIPCR2-f5 and mTIP2rev, three nested PCR products were obtained, cloned and sequenced. The largest PCR product of approximately 900 bp corresponded to the genomic DNA sequence and was therefore most likely derived from contaminating TIP39 genomic DNA or from pre-mRNA. A PCR product of approximately 650 bp lacked the intronic sequence between exons 1 and 2, while the intervening sequence between exons 1 and U1 was present. This suggested that the latter product was most likely derived from partially processed pre-mRNA. The smallest PCR product of approximately 560 bp comprised a nucleotide sequence extending from exon U1 to exon 2, which did not appear to contain intronic DNA sequences. Furthermore, no additional conserved splice sites were present in the 5′ region of this cDNA sequence, indicating that the mRNA from which this PCR product was derived had been completely processed. At least for the mouse TIP39 gene, these findings thus confirmed not only the predicted intron between exons 1 and 2, but also the intron between exons U1 and 1 that had been predicted based on the nucleotide sequence alignment of mouse and human genomic DNA clones (see FIGS. 9A and 9B, and FIG. 10). No additional differently spliced mRNAs and/or additional 5′ untranslated exons were detected.

The TIP39 sequence around the putative initiator ATG was only partially in the context of the usual consensus sequence for the initiation of translation (C\ddot{GGTG}AUGG in mouse and human; deviation from the perfect Kozak consensus is underlined) (see FIG. 9B [SEQ ID NOS: 22-29]). However, since a guanine or an adenine at position −3 and a guanine at position +4 appear to be the most important nucleotides flanking the initiator AUG (Kozak, M., Gene 234:187-208 (1999)), initiation of TIP39 translation should readily occur. An in-frame termination codon was identified 18 nucleotides upstream of the putative AUG in the mouse mRNA, but not in the human gene (see FIG. 10 [SEQ ID NO:30]).

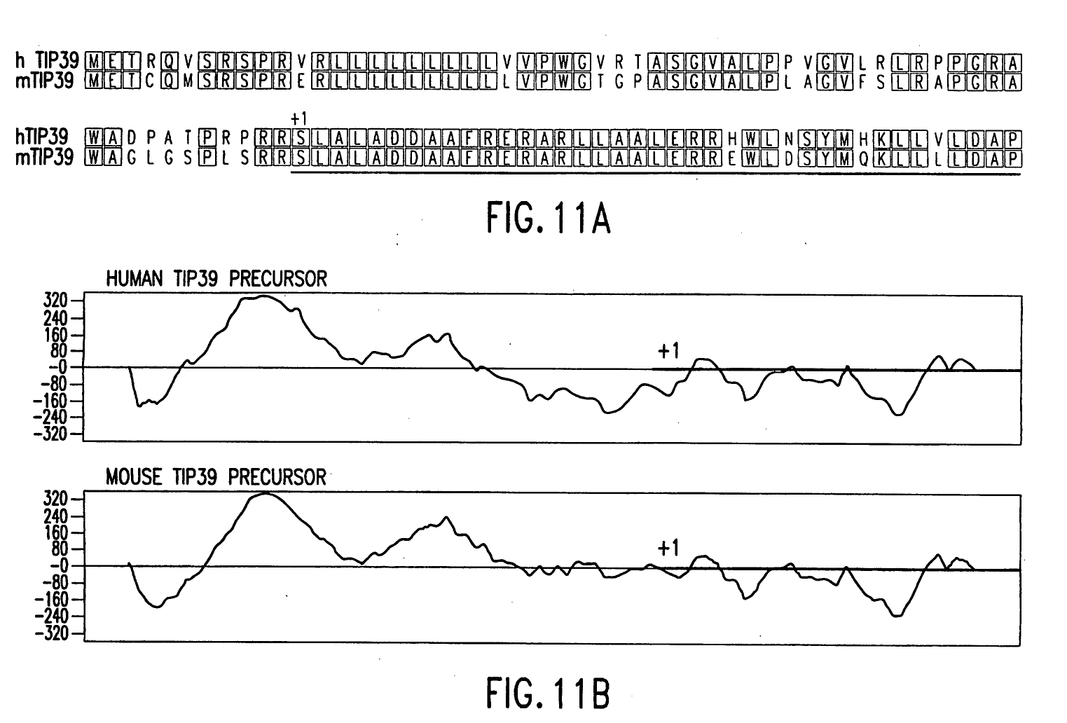

The cDNAs (Genbank accession numbers: AY048588 for human TIP39; AY048587 for mouse TIP39) encoding human and mouse TIP39 showed 80% identity across the entire coding sequences, whereas the deduced amino acid sequence was 97%/90% similar/identical for the two mature peptides. Human and mouse TIP39 precursors are both predicted to comprise 100 amino acids with an overall amino acid similarity/identity of 84%/78% (FIG. 11A)[SEQ ID NOS: 32,33]; the predicted pre-sequences alone (61 amino acids) showed less homology (77%/72% similarity/identity). The cDNAs encoding both TIP39 precursor were found to be particularly rich in guanine and cytosine (GC-content: 74.3% and 69.7%, respectively) compared to a human genome-wide average of 41% (Consortium IHGS, Nature 409:860-921 (2001)). The intervening sequences between exons U1 and 1, and between exons 1 and 2 had GC-contents of 66.6% and 59.6%, and 58.7% and 65.9%, respectively (human versus mouse). No expressed sequence tags (ESTs) derived from the human or mouse TIP39 gene were identified when searching the NCBI Genbank databases.

Comparison of the Genes Encoding TIP39, hPTH and hPTHrP

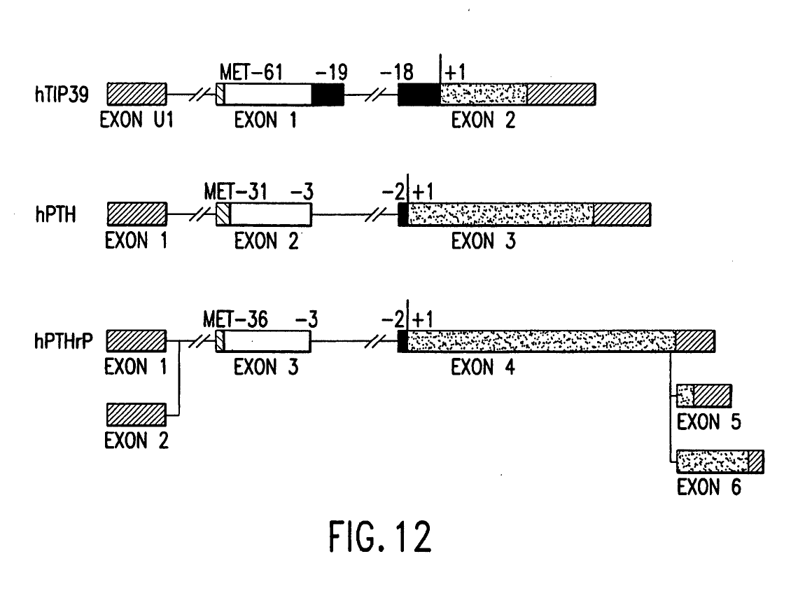

As outlined above, human and mouse TIP39 shared a high degree of structural homology. Furthermore, both genes share organizational features with the genes encoding PTH and PTHrP. Like the PTH gene (Vasicek, T., et al., Proc. Natl. Acad. Sci. USA 80:2127-2131 (1983)), TIP39 consists of at least three exons, including one exon comprising the 5′ UTR. In contrast, the PTHrP gene is more complex in that it comprises several additional coding and noncoding exons which give rise to several different mRNA transcripts (Yasuda, T., et al., J. Biol. Chem. 264:7720-7725 (1989); Yang, K. H., and Stewart, A. F., “Parathyroid hormone-related protein: the gene, its mRNA species, and protein products,” in Principles of Bone Biology, Bilezikian, J. P., et al., eds., Academic Press, New York, N.Y. (1996), pp. 347-362). While TIP39 is most likely synthesized as a longer precursor which contains an additional 61 amino acids at the amino-terminus, the precursors of PTH and PTHrP comprise shorter preprosequences (i.e. 31 and 36 amino acids, respectively). For all three genes, the message derived from the exon encoding the 5′ UTR is spliced onto the first coding exon. For PTH and PTHrP, this exon encodes all but 2 amino acids of the prepro-sequence, while the first coding exon of TIP39 encodes only about two thirds of the much longer partially hydrophobic leader sequence (i.e. amino acid residues −61 to −19) (FIG. 11B). The remaining portion of the precursor sequence is encoded by exon 2, i.e. the equivalent of the exons encoding mature PTH and PTHrP (FIG. 12).

PTH, PTHrP and TIP39 must undergo post-translational processing to yield biologically active peptides. While processing of the PTH and PTHrP precursors was previously explored (for review see: Kronenberg, H. M., et al., “Parathyroid hormone: Biosynthesis, secretion, chemistry, and action,” in Handbook of Experimental Pharmacology: Physiology and Pharmacology of Bone, Mundy, G. R. and Martin, T. J., eds., Springer-Verlag, Heidelberg, Germany (1993), pp. 185-201; Broadus, A. E., and Stewart, A. F., “Parathyroid hormone-related protein: Structure, processing, and physiological actions,” in The parathyroids. Basic and Clinical Concepts, Bilezikian, J. P., et al., eds., Raven Press, New York, N.Y. (1994), p. 259-294), there are no experimental data yet exploring the generation of mature TIP39 from its precursor. Using the neural network algorithm provided by the SignalP World Wide Web server (Nielsen, H., et al., Protein Engineering 10:1-6 (1997); Nielsen, H., et al., Protein Engineering 12:3-9 (1999)), signal peptide cleavage sites were predicted for human and mouse TIP39 between amino acid residues −32 and −31 (human residues: VRT-AS; mouse residues: TGP-AS). Using the same algorithm, the established processing sites for PTH and PTHrP were correctly predicted, making it plausible that the cleavage sites predicted for the two mammalian TIP39 molecules are indeed correct. It is currently unknown whether the TIP39 precursor contains, similar to PTH and PTHrP, a pre-sequence and a pro-sequence. However, the amino acid sequence preceding the cleavage site between the putative pro-hormone and the mature peptide contains two basic residues in both mammalian TIP39 species. These residues are typically found at the end of pro-sequences (Harris, R. B., Arch. Biochem. Biophys. 275:315-333 (1989)), including PTH and PTHrP (Yasuda, T., et al., J. Biol. Chem. 264:7720-7725 (1989); Vasicek, T., et al., Proc. Natl. Acad. Sci. USA 80:2127-2131 (1983)), and residues −31 to −1 could thus represent the pro-sequence of TIP39.

Phylogenetic Analysis of TIP39, PTH, and PTHrP

Amino acid sequence comparison of the receptors for secretin, calcitonin, PTH-PTHrP, and several other peptides of intermediate length revealed a close phylogenetic relationship, thus establishing the class B family of G protein-coupled receptors (Jüppner, H., Current Opinion in Nephrology & Hypertension 3:371-378 (1994); Rubin, D. A., and Jüppner, H., J. Biol. Chem. 84:28185-28190 (1999)). It is furthermore well established that PTH and PTHrP evolved through an ancient gene-duplication event from a common precursor (Broadus, A. E., and Stewart, A. F., “Parathyroid hormone-related protein: Structure, processing, and physiological actions,” in The parathyroids. Basic and Clinical Concepts, Bilezikian, J. P., et al., eds., Raven Press, New York, N.Y. (1994), p. 259-294). Since TIP39 shares some amino acid sequence homology with PTH and PTHrP, and since all three peptides interact not only with the PTH/PTHrP receptor, but also with the PTH2 receptor, the possibility that the genes encoding these peptides derived from a common ancestor was assessed. Amino acid sequence alignment of all known PTH and PTHrP molecules, as well as murine, bovine, and human TIP39 were aligned and analyzed by distance and parsimony methods. Several secretin and VIP species were included in the analysis, since these peptides share within the amino acid sequences of their signal and secreted peptides several parsimony-informative characters with PTH, PTHrP, and TIP39. Phylogenetic inference furthermore strongly suggested that human GIP can be used an appropriate outgroup (Sherwood, N., et al., Endocrine Reviews 21:619-670 (2000); Swofford, D., et al., “Phylogenetic inference,” in Molecular Systematics, Hillis, D, et al., eds., Sinauer Associates, Inc., Sunderland, Mass. (1996), pp. 407-514).

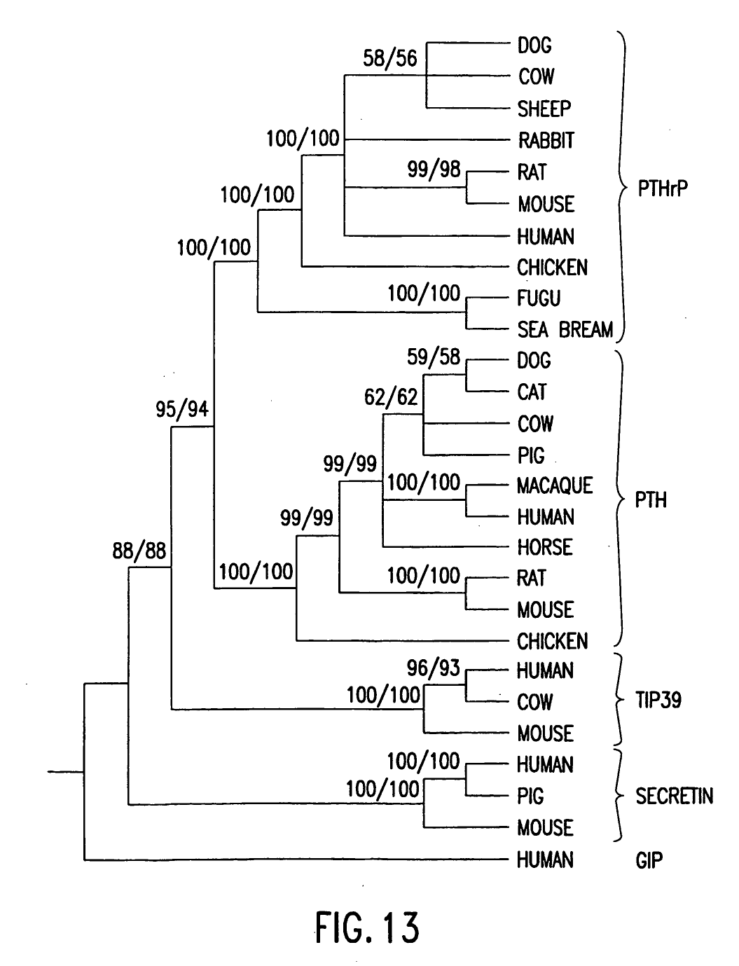

Although the terminal branches differed depending on whether Maximum Parsimony or Neighbor-Joining analyses were performed, all trees showed the same topology of groups, i.e. distinct groups for PTH, PTHrP, TIP39, and secretin. The highest bootstrap and jackknife values were obtained by Neighbor-Joining analysis when including the full-length precursor proteins, which included the signal peptides (FIG. 13); present theories indicate that nodes with bootstrap values above 95% are considered strongly supported of a close phylogenetic relationship (Page, R., and Holmes, E. Molecular Evolution: a phylogenetic approach, Blackwell Science Ltd., Oxford, UK (1998); Felsenstein, J., and Kishino, H., Syst. Biol. 42:193-200 (1993)). Although minor differences in the phylogenic relationship within the clades for PTH and PTHrP could not be resolved due to the limited amount of characters available for analysis, significant phylogenic differences in the relationships between PTH, PTHrP and TIP39 groups became apparent. Consistent with the previously established gene duplication event (Broadus, A. E., and Stewart, A. F., “Parathyroid hormone-related protein: Structure, processing, and physiological actions,” in The parathyroids. Basic and Clinical Concepts, Bilezikian, J. P., et al., eds., Raven Press, New York, N.Y. (1994), p.259-294), the results indicated that PTH and PTHrP belong to closely related sister groups. Furthermore, even though the precursor sequence was not available for bovine TIP39, which may alter the overall significance values, TIP39 grouped strongly as the sister group to the PTH-PTHrP superfamily, implying that all three groups of ligands are derived from a common precursor. However, the isolation of peptides with similarities to PTH, PTHrP, TIP39 from lower vertebrate species will be required to confirm this hypothesis.

Characterization of the TIP39 Gene Product

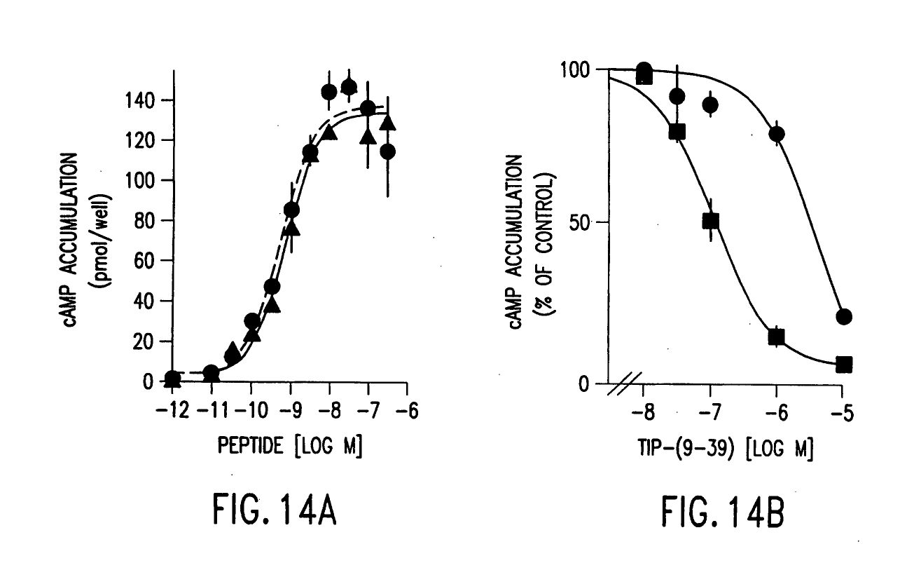

To determine whether mouse and human TIP39 (which is identical to bovine TIP39) activate the human PTH2 receptor with similar efficiency, both peptides were synthesized and agonist-induced cAMP accumulation in LLCPK₁ cells stably expressing this receptor was assessed. Both peptides showed equal potency and efficacy at this receptor (EC₅₀ for human TIP39: 0.54 nM; EC₅₀ for mouse TIP39: 0.74 nM; maximum cAMP accumulation: 136.5±4.9 pmol/well and 133.7±3.9 pmol/well, respectively) (FIG. 14A). Analogs of TIP39 were recently shown to be potent inhibitors of PTH-(1-34) action at the PTH/PTHrP receptor (Jonsson, K. B., et al., Endocrinology 142:704-709 (2001); Hoare, S. R., et al., J. Biol. Chem. 275:27274-27283 (2000); Hoare, S. R. J., and Usdin, T. B., J. Pharmacol. Exp. Ther. 295:761-770 (2000)). We therefore tested whether TIP-(9-39) can antagonize the actions of PTH-(1-34) and human TIP39 at the PTH2 receptor. The actions of either agonist, at concentrations that induced half-maximal cAMP accumulation in cells expressing the PTH2 receptor were inhibited by TIP-(9-39). The activity of 1 nM PTH-(1-34) was inhibited by TIP-(9-39) with an IC₅₀ of 1.1×10⁻⁷ M, while approximately 14-fold higher concentrations of the antagonist were required to antagonize cAMP accumulation stimulated by 1 nM human TIP39 (IC₅₀: 4×10⁻⁶ M) (FIG. 14B). The efficacy of TIP-(9-39) as an antagonist at the PTH2 receptor thus appears to be similar to that of [Nle^{8,18}, D-Trp¹², Tyr³⁴]-bPTH-(7-34)NH₂ and [Leu¹¹, D-Trp¹²]hPTHrP-(7-34)NH₂ at the PTH/PTHrP receptor (Behar, V., et al., Endocrinology 137:2748-2757(1996)), which may be sufficient to help exploring the biological roles of TIP39.

TIP39 Expression

Transcripts encoding the human PTH2 receptor were initially detected by Northern blot analysis in poly-A⁺ RNA from brain, pancreas, testis, and placenta (Usdin, T. B., et al., J. Biol. Chem. 270:15455-15458 (1995)). In situ hybridization studies subsequently revealed mouse mRNA transcripts in glomeruli, somatostatin synthesizing D cells of the pancreatic islets, and numerous areas of the brain, including the preoptic area of the periventricular nucleus, the diagonal band of Broca, the amygdala, the arcuate nucleus, ventromedial nucleus and dorsal paraventricular nucleus among other areas (Usdin, T. B., et al., Endocrinology 137:4285-4297 (1996); Wang, T., et al., Neuroscience 100:629-649 (2000); Usdin, T. B., et al., Frontiers in Neuroendocrinology 21:349-383 (2000)).

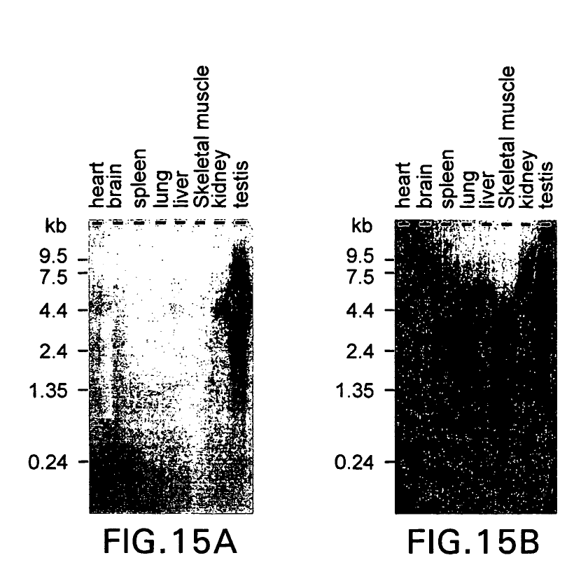

Northern analysis using a blot with poly-A⁺ RNA (2 μg/lane) from multiple mouse tissues revealed a prominent message of approximately 4.5 kb in testis, which was also observed, albeit at much lower intensity, in liver, kidney, possibly heart (FIG. 15). Poly-A⁺ RNA from testis furthermore revealed two larger transcripts, while poly-A⁺ RNA from liver showed evidence for very weakly hybridizing transcripts of about 1.5 kb, and poly-A⁺ RNA from brain showed transcripts of 1.0 kb and possibly 0.7 kb. With the exception of testis, TIP39 does not seem to be abundantly expressed. The presence of TIP39 mRNA transcripts that are different in size suggests that its gene may comprise more exons than currently known. It is also plausible that TIP39 comprises a longer 3′ non-coding region than suggested by the presence of a consensus polyadenylation signal just down-stream of the termination codon, or that the hybridizing mRNA in testis represents incompletely processed pre-mRNA.

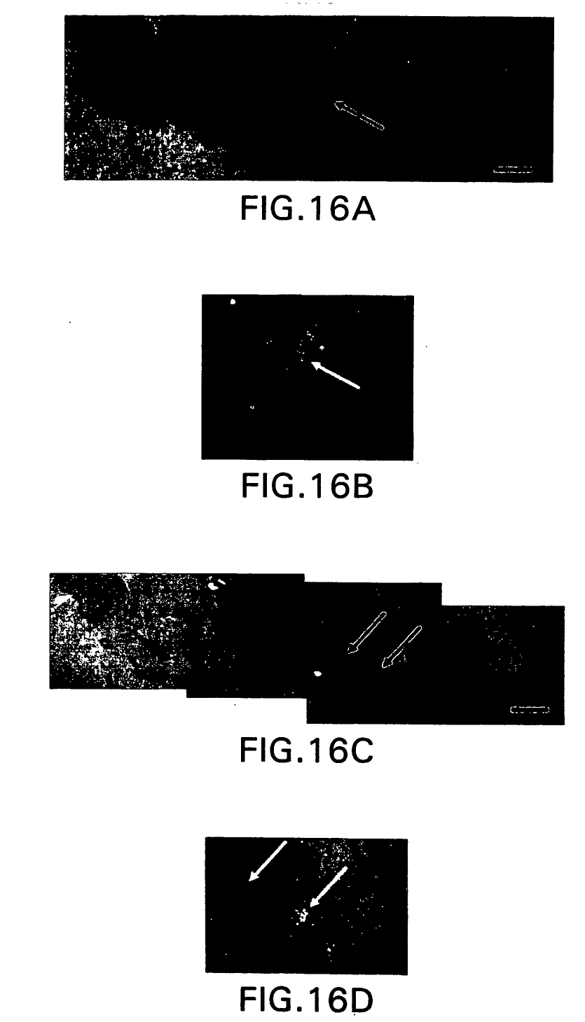

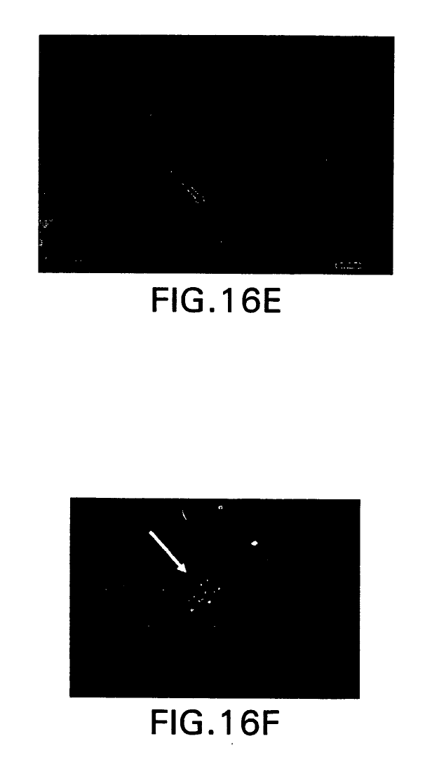

To assess TIP39 expression further, in situ hybridizations were performed using those two tissues that express the PTH2 receptor most abundantly (e.g. brain and testis) and may thus represent targets of this peptide. Specific hybridization was detected in consecutive sections of adult mouse brain (FIG. 16A-16F), particularly within focal areas corresponding to the nucleus ruber and the nucleus centralis pontis, both of which have been implicated in the regulation of motor activity, and in the nucleus subparafascicularis thalami, which has been implicated in nociception. This distribution is different from the in situ data reported for the mRNA encoding the PTH2 receptor (Usdin, T. B., et al., Endocrinology 137:4285-4297 (1996); Wang, T., et al., Neuroscience 100:629-649(2000)), suggesting that TIP39 synthesis and secretion occurs distant from its site(s) of action. However, immunohistochemical studies may be necessary to determine whether TIP39— apart from possible paracrine/autocrine roles—is neuronally transported from the cerebral nuclei where it is synthesized to those areas of the brain, where the PTH2 receptor is expressed. Overall, in situ hybridizations and Northern blot analysis suggested that TIP39 is expressed in mice in only few tissues.

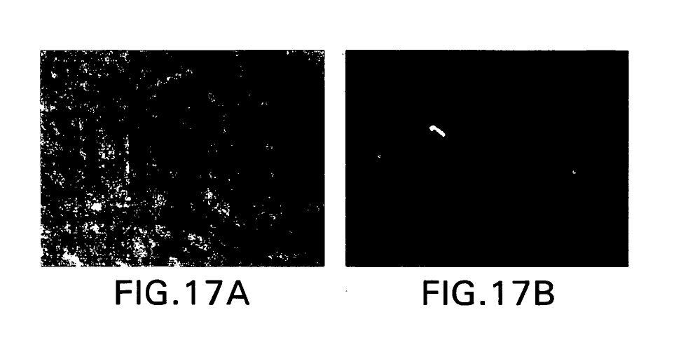

Most prominent TIP39 mRNA expression was detected in the epithelium of seminiferous tubules (FIG. 17). Analysis of the expression pattern suggested marked stage-specific differences. However, further studies are needed to assess the differentiation stage of those tubule segments expressing TIP39 mRNA. In contrast to these findings, PTH2 receptor expression in testis was reported to occur in the interstitium between spermatic tubules, i.e. in Leydig cells, as well as in sperm and within the epididymis (Usdin, T. B., et al, Endocrinology 137:4285-4297 (1996)). Taken together, these findings suggest that TIP39 and the PTH2 receptor may have a role in cAMP generation in seminiferous tubules and could thus have, similar to PACAP (Daniel, P. B., and Habener, J. F., Endocrinology 141:1218-1227 (2000)), a role in spermatogenesis. No hybridization of TIP39 mRNA was detected in pancreas, where the PTH2 receptor is also expressed (data not shown).

The foregoing specification, including specific embodiments and examples is intended to be illustrative of the present invention and is not to be taken as limiting. It will be appreciated to those skilled in the art that the invention can be performed within a wide range of equivalent parameters of composition, concentration, modes of administration, and conditions without departing from the spirit or scope of the invention or any embodiment thereof. All publications, patents and patent applications cited herein are incorporated by reference in their entirety into the present disclosure.

What is claimed is:

1 An isolated polypeptide consisting of the amino acid sequence AFRERARLLAALERRHWLNSYMHKLLVLDAP. [SEQ ID No.:1] or ALADDAAFRERARLLAALERRHWL NSYMHKLLVLDAP. [SEQ ID No.:2]

2. An isolated polypeptide comprising an amino acid sequence selected from the group consisting of AFRERARLLAALERRHWLNSYMHKLLVLDAP. [SEQ ID No.:1] ALADDAAFRERARLLAALERRHWLNSYMHKL LVLDAP. [SEQ ID No.:2], AAFRERARLLAALERR HWLNSYMHKLLVLDAP [SEQ ID No.:3], FRERARLLAALERRHWLNSYMHKLLVLDAP [SEQ ID No.:4], AFRERARLLAALERRHWLNSYMHKLLVLDAP. [SEQ ID No.:1]. RERARLLAALERRHWLNSYMHKLLVLDAP [SEQ ID No.:5] and ERARLLAALERRHW LNSYMHKLLVLDAP [SEQ ID No.:6], wherein said polypeptide sequence is not TIP7-39.

3. The isolated polypeptide of claim 1 or 2, wherein there is a single amino acid substitution.

4. The isolated polypeptide of claim 1 or 2 wherein there is one or more conservative amino acid substitutions.

5. An isolated nucleic acid sequence encoding the polypeptide of claim 1 or 2.

6. An isolated nucleic acid sequence, wherein said sequence is at least 95% identical or binds under stringent conditions to the sequence of claim 3.

7. A recombinant host cell comprising the DNA of claim 5.

8. A recombinant vector comprising the DNA of claim 5.

9. An isolated polypeptide, wherein said polypeptide is a truncated polypeptide of TIP39 (SLALADDAFRERARLLAALERRH LNSYMHKLLVLDAP) [SEQ ID No.:7] and said truncated polypeptide is not TIP7-39.

10. A method for treating a mammalian condition wherein said condition is characterized by requiring antagonism of PTH1R or PTH2R, said method comprising:
a) administering to a patient in need of antagonism of PTH1R or PTH2R, an effective dose of the polypeptide of claim 1 or 2; and
b) antagonizing PTH1R or PTH2R.

11. The method of claim 10, wherein said effective amount of polypeptide antagonizing PTH1R or PTH2R is administered by providing to the patient DNA encoding said polypeptide and expressing said polypeptide in vivo.

12. The method of claim 10, wherein said condition requiring antagonism of PTH1R or PTH2R is hyperparathyroidism or hypercalcemia.

13. An isolated polypeptide comprising the sequence AFRERARLLA, wherein said sequence is not that of the polypeptide TIP39 or TIP7-39 and said isolated polypeptide binds to PTH1R or PTH2R.

14. A PTH1R or PTH2R antagonist comprising a truncated TIP39 polypeptide wherein said antagonist is not TIP7-39.

15. The PTH1R or PTH2R antagonist of claim 14 wherein said antagonist is TIP3-39 or TIP9-39.

16. The PTHR1 or PTH2R antagonist of claim 14, wherein said antagonist has an apparent binding affinity at least 2-fold higher than TIP1-39.

17. The PTHR1 or PTH2R antagonist of claim 14, wherein said antagonist has an apparent binding affinity at least 3-fold higher than TIP1-39.

18. The PTHR1 or PTH2R antagonist of claim 14, wherein said antagonist has an apparent binding affinity at least 5-fold higher than TIP1-39.

19. A PTH1R agonist comprising a sequence of the chimeric polypeptide selected from the group of sequences consisting of PTHrP(1-20)/TIP(23-39) (AVSEHQLLHDKGKSI QDLRR RHWLNSYMHKLLVLDAP) [SEQ ID NO:12], PTHrP(1-9)/TIP(12-39) (AVSEHQLLH ERARLLAALER RHWLNSYMHKLLVLDAP) [SEQ ID NO:13] and PTHrP(1-13)/TIP(16-39)(AVSEHQLLHDKGK LLAALER RHWLNSYMHKLLVLDAP) [SEQ ID NO:14].

20. A method for treating mammalian conditions characterized by increases in calcium resulting from excess PTH or PTHrP comprising:
a) administering to a patient in need thereof an effective dose of the polypeptide of claim 1 or 2; and
b) antagonizing PTH1R or PTH2R.

21. A method for treating mammalian conditions characterized by decreases in bone mass, wherein said method comprises administering to a subject in need thereof an effective bone mass-increasing amount of the polypeptide of claim 19.

22. A method for treating mammalian conditions characterized by an abnormality related to the activated PTH2R.

23. An antibody against a polypeptide of claim 1 or 2.
